(19) United States
(12) Patent Application Publication
(10) Pub. No.: US 2026/0048188 A1
(43) Pub. Date: Feb. 19, 2026

(54) ATTRIBUTE-SENSING PERITONEAL DIALYSIS SYSTEMS AND ASSOCIATED METHODS

(71) Applicant: POWELL MEDICAL INNOVATIONS, LLC, San Antonio, TX (US)

(72) Inventor: Jacob Alexander Powell, San Antonio, TX (US)

(21) Appl. No.: 18/873,451

(22) PCT Filed: Jun. 20, 2023
(86) PCT No.: PCT/US2023/025771

§ 371 Date: Dec. 10, 2024

Related U.S. Application Data
(60) Provisional application No. 63/353,713, filed on Jun. 20, 2022.

(51) Int. Cl. A61M 1/28

(52) U.S. Cl. CPC A61M 1/287; A61M 1/285; A61M 2202/0014; A61M 2205/3327; A61M 2205/50; A61M 2230/201; A61M 2230/208

(57) ABSTRACT

Devices and associated methods and systems for measuring one or more attributes of a waste fluid during peritoneal dialysis using a sensor assembly coupled to a drain line or a waste receptacle. The sensor assembly may receive a portion of a waste fluid, via capillary force or absorption utilizing one or more of microchannels and sensing material. A sensor may be disposed adjacent the microchannels or sensing material to measure one or more attributes of the waste fluid. The sensor assembly may also include a second portion that includes a battery, microprocessor, and a transmitter. The sensor assembly may process the measured attributes and then, transmit the processed attributes to a computer.

### PRIORITY CLAIM

This application claims the benefit of priority to U.S. Provisional Application No. 63/353,713, filed Jun. 20, 2022, the benefit of which is claimed and the disclosure of which is incorporated by reference in its entirety.

### FIELD

The present disclosure generally relates to a peritoneal dialysis system, and more specifically, to an attribute-sensing peritoneal dialysis system and associated methods.

### BACKGROUND

Humans must have waste products removed from their blood to live healthy lives. When a person's renal function is inadequate, dialysis provides a way to remove waste from the blood. There are two main types of dialysis: hemodialysis and peritoneal dialysis (PD). Hemodialysis is more commonly known and externally filters blood through a dialysis machine and returns the filtered blood back to the body.

With the second type, peritoneal dialysis, the blood is cleaned inside a patient's body, not outside as with hemodialysis. There are two types of peritoneal dialysis that vary mainly with respect to exchanges. The first is continuous ambulatory peritoneal dialysis (CAPD), and the second is automated peritoneal dialysis (APD). With CAPD, no machine is involved. Instead, dialysate is put into the peritoneal cavity and allowed to dwell for a set time. Then it is removed through the catheter to the drainage bag. APD is the same except a machine, called a cycler, is used to deliver the dialysate and then drain the waste fluid.

During PD, the cleansing fluid, or dialysate, is circulated through a tube or catheter inside part of the patient's peritoneal cavity, which is part of the patient's abdominal cavity. The dialysate absorbs waste products from blood vessels in the patient's peritoneum during a dwell and then after a certain amount of time the dialysate is drawn back out of the body and discarded. The portion drawn backout goes through a drainage line into a drainage bag or toilet. In the process of PD, the lining of the patient's peritoneum functions as a semipermeable membrane that allows diffusion and osmosis exchanges to take place between the dialysate and the bloodstream. This exchange allows for the removal of waste products from the blood.

Many patients prefer PD to hemodialysis because PD is usually done at home. However, recent studies have shown that a majority of PD patients have to switch to hemodialysis to ensure efficient dialysis. One issue with conventional peritoneal dialysis is that because the treatment is done at home, changes to the membrane may not be detected as quickly, as these changes would be detected in a clinical setting. Additionally, if the changes to the membrane are not detected, then the patient is likely to maintain the same treatment, which can result in excessive exposure to high glucose concentrations resulting in membrane failure and ultrafiltration failure.

### SUMMARY

In one or more example embodiments, a sensor assembly for use during Peritoneal Dialysis is provided. The sensor assembly includes a first cell. The first cell includes a plurality of microchannels and a plurality of sensors. The plurality of microchannels include: a first microchannel, having a first end and an opposing, second end; and a second microchannel, parallel to the first microchannel and having a third end and an opposing, fourth end. The plurality of sensors include: a first sensor disposed at the second end of the first microchannel; and a second sensor disposed at the fourth end of the second microchannel.

In another example embodiment, a peritoneal dialysis system is provided. The peritoneal dialysis system includes a catheter configured for insertion in a peritoneum of a patient; a sensor assembly fluidly coupled to the catheter; a dialysate bag fluidly coupled to the catheter. The sensor assembly includes a first portion having a flow path defined therethrough; and a second portion detachable from the first portion. The first portion has a first cell disposed along a first path, the first cell having at least one microchannel.

In at least one example embodiment, a peritoneal dialysis method is provided. The peritoneal dialysis method includes inserting a catheter into a peritoneum of a patient; introducing, via the catheter, dialysate from a dialysate bag into the peritoneum; removing, via the catheter, waste fluid from the peritoneum into a drain line; and measuring, via a sensor assembly, during removal of the waste fluid, at least one electrolyte concentration of a first portion of the waste fluid.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. In addition, the present disclosure may repeat reference numerals, letters, or both in the various examples. This repetition is for the purpose of simplicity and clarity and does not in itself dictate a relationship between the various embodiments and/or configurations discussed.

FIG. 1 is a diagrammatic illustration of a peritoneal dialysis (PD) system in accordance with one or more embodiments.

FIG. 2 is a block diagram depicting a sensor assembly of the PD system of FIG. 1 in accordance with one or more embodiments.

FIG. 3A is an illustration of the sensor assembly of the PD system of FIG. 2 with one or more embodiments.

FIG. 3B is another illustration the sensor assembly of the PD system of FIG. 2 in accordance with one or more embodiments.

FIG. 4 is a diagrammatic illustration of a cross-section of the housing of FIG. 3B in accordance with one or more embodiments.

FIG. 5 is flowchart of a method for attaching a sensor assembly to a drain line in accordance with one or more embodiments

FIG. 6A is a diagrammatic illustration of constructing a sensor assembly in accordance with one or more embodiments.

FIG. 6B is a diagrammatic illustration of inserting the sensor assembly of FIG. 6A in a housing in accordance with one or more embodiments.

FIG. 6C is a diagrammatic illustration of coupling the sensor assembly of FIG. 6B to a drain line in accordance with one or more embodiments.

FIG. 6D is a diagrammatic illustration of the sensor assembly of FIG. 6C coupled to the drain line in accordance with one or more embodiments.

FIG. 7 is diagrammatic illustration of the sensor assembly of FIG. 6C in accordance with one or more embodiments.

FIG. 8 is another diagrammatic illustration of the sensor system including the sensor assembly of FIG. 7 in accordance with one or more embodiments.

FIG. 9 is flowchart of a method for sensing various attributes during PD in accordance with one or more embodiments.

FIG. 10 is another diagrammatic illustration of the peritoneal dialysis (PD) system of FIG. 1 in accordance with one or more embodiments.

FIG. 11 is an illustration of a sensor assembly disposed on a waste receptacle of the PD system of FIG. 10 in accordance with one or more embodiments.

FIG. 12 is another illustration of the sensor assembly of FIG. 11 in accordance with one or more embodiments.

FIG. 13 is an illustration of a cross-sectional view of the sensor assembly disposed on the waste receptacle of FIG. 11 in accordance with one or more embodiments.

FIG. 14 is an illustration of yet another view of the sensor assembly disposed on a waste receptacle of FIG. 10 in accordance with one or more embodiments.

FIG. 15 is a cross-sectional illustration of the sensor assembly of FIG. 10 disposed within a rigid support structure in accordance with one or more embodiments.

### DETAILED DESCRIPTION

Illustrative embodiments and related methods of the present disclosure are described below as they might be employed in one or more methods and systems for attribute sensing during peritoneal dialysis. In the interest of clarity, not all features of an actual implementation or method are described in this specification. It will of course be appreciated that in the development of any such actual embodiment, numerous implementation specific decisions must be made to achieve the developers' specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure. Further aspects and advantages of the various embodiments and related methods of the disclosure will become apparent from consideration of the following description and drawings.

The example embodiments described below recognize that it may be desirable to have methods and systems that more efficiently and more consistently monitor patients who use peritoneal dialysis (PD). In particular, the embodiments described below provide methods and systems for: (1) monitoring various attributes while performing PD at the patient's home, (2) allowing the patient and/or medical professional access to the monitored attributes in real-time, (3) providing optimized treatment plans as a result of the monitored attributes, and/or (4) tracking the patient's medical progress and/or treatment over time. Therefore, the methods and systems provide an improvement to the technical field of PD systems by providing remote testing and monitoring.

One or more embodiments described below provide methods and systems for optimizing the patient's PD treatment throughout a year. For example, conventionally, a PD patient may be subjected to a peritoneal equilibrium test, which tests the peritoneal membrane transport status in a patient. This test typically is done only once a year because it is so time intensive. However, having infrequent assessments increases the risk of receiving improper treatment. Therefore, one or more embodiments described herein provide methods and systems for optimizing the patient's PD treatment by providing patients and/or medical professionals (such as nurses, doctors, physician's assistants, medical receptionists, and the like) with data throughout the year. By receiving data on a more consistent basis, the patient may receive more optimized care and may stay on PD longer where optimized care refers to optimized glucose concentration in the dialysate fluid and optimized dwell time for the dialysis fluid in the peritoneum. By receiving data consistently, medical professionals may determine that this patient needs to switch treatments and notify the patient of the change in treatment. The methods and systems described herein advantageously provide information for disease management, specifically regarding the management of transport status and metabolic status.

One or more illustrative embodiments described below provide methods and systems for constructing a sensor assembly and attaching the sensor assembly to a drainage line. The methods and systems described herein advantageously provide a sensor that measures waste fluid during PD and may transmit the sensed (measured) attributes to a smart device and/or database during PD.

One or more illustrative embodiments described below provide methods and systems for using microfluidic channels (microchannels) to move a portion of the waste fluid from either a drain line or a drainage bag to a sensor mounted at the end of the microchannel. The sensor may be a potentiometric sensor, electrochemical sensor, colorimetric sensor, pH sensor, and the like. Each microchannel may have a different type of sensor disposed at the end of the microchannel to detect a variety of attributes in the portion of the waste fluid. The sensor may detect concentration and/or levels of potassium, glucose, magnesium, sodium, pH, calcium, phosphate, urea, creatine, blood urea nitrogen (BUN), bicarbonate, lactate, and other diagnostic features. The sensor may even detect hue changes if a colorimetric sensor was used. The methods and systems described herein advantageously provide a way to measure or detect a variety of attributes in the waste fluid.

One or more embodiments described below provide a graphical user interface to display the data from the sensor, the treatment plan, and messages from the medical professionals to the patient. This interface may graphically chart the measured attributes over time. The interface may send an alert to the patient if one or more of the attributes are outside threshold levels. The methods and systems described herein advantageously provide an interface that includes real-time monitoring of a patient's PD treatment.

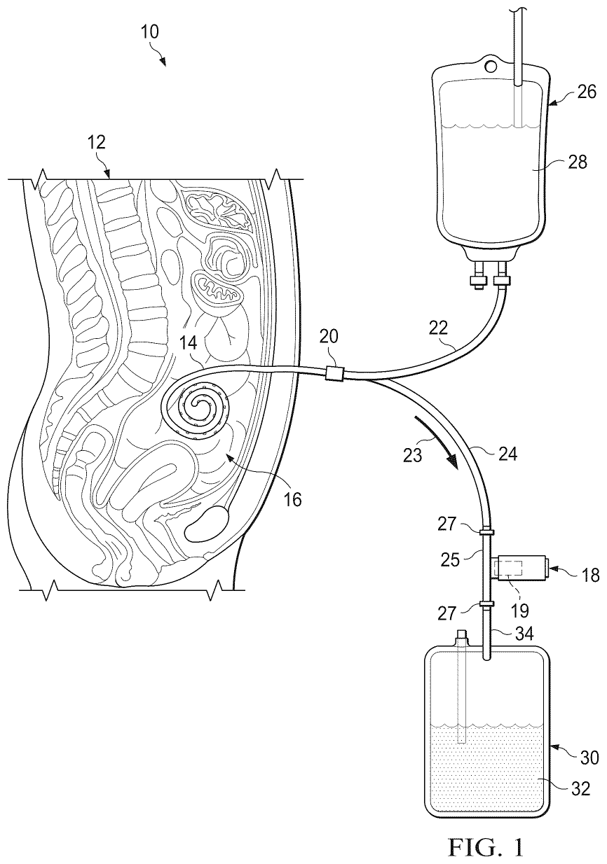

Referring to FIG. 1, an embodiment of a peritoneal dialysis (PD) system 10 is shown. A patient 12 is illustrated in partial cross section with a catheter 14 inserted at least partially within the patient's peritoneum 16. The catheter 14 is in fluid communication with a sensor assembly 18 having one or more sensor 19. In one or more embodiments, the catheter 14 is coupled to a connector 20 which is in fluid communication with a supply line 22 and a waste line 24. The supply line 22 is in fluid communication with a dialysis solution bag, or dialysate bag 26, which contains a dialysis solution or dialysate 28. The waste line 24 may be in fluid communication with a peritoneal dialysis waste fluid vessel 30 in the form of a waste receptacle, such as a bag that collects waste fluid 32 or a toilet. A drainage line 34 may be extended from the sensor assembly to the waste receptacle 30. Any of the foregoing lines 22, 24, 34 may be flexible tubing as is known in the industry. As used herein, waste fluid vessel refers to any structure that can contain waste fluid, either for collection or as a flow stream.

In some embodiments, the PD system 10 includes a cycler operationally coupled to the catheter 14.

In one or more embodiments, the sensor assembly 18 is disposed along the waste flow path 23 of the waste fluid 32 flowing from catheter 14. In one or more embodiments, sensor assembly 18 may be disposed upstream of waste receptacle 30, while in other embodiments, sensor assembly 18 may be disposed in or form a part of waste receptacle 30. In the illustrated embodiment, sensor assembly 18 is shown upstream of waste receptacle 30, disposed along the waste flow path 23 between waste line 24 and drainage line 34. In one or more embodiments, the sensor assembly 18 includes a bridge tubing 25 that can be inserted with connectors 27 along waste line 24 or drainage line 34, enabling sensor assembly 18 to be readily disposed along the waste flow path 23. In some embodiments, sensor 19 of sensor assembly 18 may include one or more metal-based sensor(s), including but not limited to a potentiometric sensor. In some embodiments, the potentiometric sensor is formed using one or more ion-selective optodes (ISE) or other metal-based carrier, which may provide an electric signal in response to various concentrations of the target attributes (i.e., analytes) or other characteristics. In one or more embodiments, sensor 19 of sensor assembly 18 may include one or more non-metal-based sensor(s), including but not limited to colorimetric or color changing sensors such as ion-selective optodes (ISO) on a carrier, such as paper, plastic or other flexible material. In some embodiments, the sensor assembly 18 includes a plurality of sensors 19. In some embodiments, sensor 19 of sensor assembly 18 may include a potentiometric sensor and a colorimetric sensor. In some embodiments where sensor 19 is an ISO sensor, sensor assembly 18 may include a hue measuring device as discussed below.

In some embodiments, the supply line 22 forms a portion of a drainage line 34. In some embodiments, the waste line 24 forms a portion of a drainage line 34. In some embodiments, the supply line 22 and the waste line 24 are tubing.

In some embodiments, the waste receptacle 30 is omitted. In some embodiments, the waste receptacle 30 is a toilet. In other embodiments, the waste receptacle 30 is a waste fluid bag, such as is shown in FIG. 1.

In operation, the dialysate 28 flows from the dialysate bag 26 through the supply line 22 through the catheter 14 into the patient's peritoneum 16. When the dialysate 28 is in the body of the patient, the solution absorbs waste and extra fluid from the body. Then, the waste fluid 32 is drained using the catheter 14 and waste line 24. As the waste fluid 32 is drained, a portion of the waste fluid 32 is exposed to sensor assembly 18 for detection and measuring. The sensor assembly 18, which is coupled to the drainage line 34, may detect a variety of attributes such as, but not limited to: glucose, sodium, calcium, magnesium, and/or phosphate. The remainder of the waste fluid 32 flows out of the drainage line 34 to waste receptacle 30.

In one or more embodiments, the sensor assembly 18 measures various attributes of the waste fluid 32. In some embodiments, the sensor assembly 18 measures electrolytes and/or the pH of the waste fluid 32. In some embodiments, the attributes measured by the sensor assembly 18 includes potassium, glucose, magnesium, sodium, pH, calcium, phosphate, and the like. In some embodiments, the sensor assembly 18 measures electrolyte concentration and/or glucose levels. In some embodiments, the sensor assembly 18 may measure sodium ion concentration of dialysis effluent (100-200 mmol/L, 1 mmol resolution), potassium ion concentration of dialysis effluent (0-10 mmol/L, 1 mmol/L), calcium ion concentration of dialysis effluent (0-10 mmol/L, 1 mmol/L), phosphate ion concentration of dialysis effluent (0-20 mmol/L, 1 mmol/L), and glucose concentration of dialysis effluent (0-250 mmol/L, 1 mmol/L). Other attributes of the waste fluid 32 may be detected in other embodiments. In some embodiments, sodium measurements may establish a baseline for measurement of potassium in the case of cross reactivity of sodium with other ionophores. In some embodiments, incorporation of other readings may be used including urea, creatine, blood urea nitrogen (BUN), bicarbonate, lactate, and other diagnostic features. For example, creatinine and BUN or other attributes (or analytes) of interest may be detected for the purpose of replicating a Peritoneal Equilibration Test and/or for the measurement of Kt/V, a common metric for measuring the filtration rate of a patient's peritoneum. In some embodiments, a different sensing modality is needed within sensor assembly 18 that is not an ISO or ISE, such as but not limited to enzyme-linked immunoassay (ELISA), Immunofluorescence, Western Blot, or Antibody testing-such as for testing creatinine, BUN, lactate, or other proteins. In some embodiments, bicarbonate and lactate are attributes of interest due their potential in assessing a patient's acid-base status. In some embodiments, sensor assembly 18 measures bicarbonate using either an ISO or ISE.

In one or more embodiments, the sensor assembly 18 produces quantitative values. In some embodiments, a cation sensor may measure potassium, sodium, and/or calcium. In one or more embodiments, an electrochemical method is used by the sensor assembly 18 to measure the cations. In some embodiments, when particles of the waste fluid 32 travel through an ion selective membrane such as the ISE or react with a highly selective reagent, the sensor assembly 18 is no longer in equilibrium; therefore, a change of potential or current will be introduced within the sample. Compact ISEs, in some embodiments, provide a way to measure cations such as sodium, potassium, calcium, and the like, such that when one type of ions travels through a barrier that only allows for diffusion of this type of ion, different concentrations of the ions on each side will generate a potential difference. Potential difference is linearly correlated to concentration. In some embodiments, ISE will read a value of 0 if no waste fluid 32 has traveled through the sensor assembly 18. In one or more embodiments, ISE will read a number above 0 when waste fluid 32 has traveled through the sensor assembly 18.

In some embodiments, an electrochemical method is used by the sensor assembly 18 to measure glucose. In some embodiments, glucose concentration is quantified by measuring current while glucose in the waste fluid 32 is reacting with glucose oxidase, and the current value is linearly correlated to glucose concentration. In some embodiments, a non-electrolyte sensor is used to measure glucose such as a non-direct enzymatic sensor. In some embodiments, the sensor assembly 18 measures differences in dialysate 28 and serum electrolyte values producing a lagging measure of plasma electrolyte concentrations.

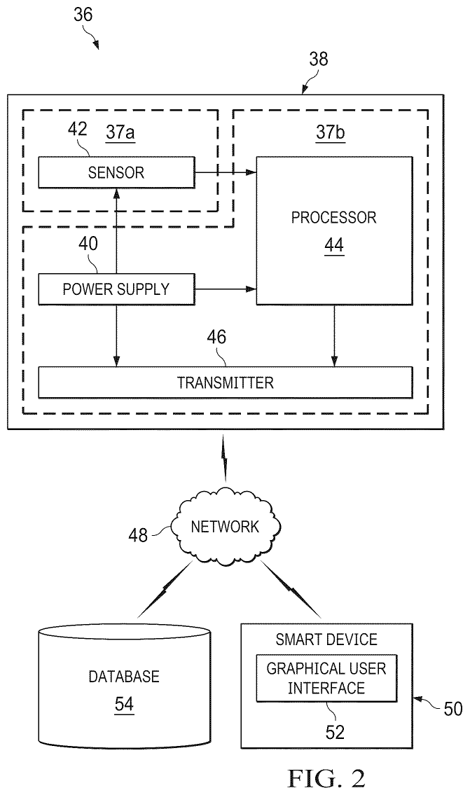

Referring to FIG. 2, one embodiment of the sensor assembly 18 is shown in more detail and is generally referred to by reference numeral 36 and includes a support housing 38. The support housing 38 as described is a rigid or semi-rigid structure or frame that supports one or more of a power supply 40, a sensor 42, a processor 44, and a transmitter 46. The power supply 40 is in communication with and/or operably coupled to the sensor 42, the processor 44, and the transmitter 46. The sensor 42 is in communication with and/or operably coupled to the power supply 40 and the processor 44. The processor 44 is in communication with and/or operably coupled with the power supply 40, the sensor 42, and the transmitter 46. The transmitter 46 is in communication with and/or operably coupled to the processor 44 and the power supply 40. The PD system 10 includes the transmitter 46 in communication, via a network 48, with a smart device 50. The smart device 50 includes a graphical user interface 52. The smart device 50 and/or the transmitter 46 may be in communication with a database 54 via the network 48.

In operation, the power supply 40 supplies power to the sensor 42, the processor 44, and the transmitter 46. The sensor 42 detects and measures levels of concentration of attributes and transmits the data to the processor 44 for processing. The processor 44 then transmits the processed data to the transmitter 46. The transmitter 46 sends the processed data, via the network 48, to the database 54 and/or to the smart device 50. The smart device 50 displays the processed data on the graphical user interface 52.

In some embodiments, the sensor assembly 36 includes two parts—a disposable portion 37a and a non-disposable portion 37b. The sensor 42, in some embodiments, may be the disposable portion 37a that is changed after each test, while the power supply 40, the processor 44, and the transmitter 46 may be the non-disposable portion 37b. The disposable portion 37a may be directly coupled to a drainage line, such as drainage line 34. Likewise, in some embodiments, the disposable portion 37a may be coupled to a waste receptacle 30, such as a trainable bag.

In some embodiments, the power supply 40 is one or more batteries. In other embodiments, the power supply 40 receives power from a device outside of the housing 38. In some embodiments, the power supply 40 includes a power cord that may be electrically coupled to an outside charging device or power outlet.

In one or more embodiments, the sensor 42 may be a potentiometric sensor, such as an ISE, that includes a working electrode and a reference electrode. The power supply 40 may apply a constant electric current to be applied to the potentiometric sensor. The potentiometric sensor measures the potential difference between the working electrode and the reference electrode, which is the voltage difference between the working electrode and the reference electrode. The reference electrode may be used to establish a baseline. When the voltage difference occurs, an electrolyte compound of the waste fluid 32, which is between the two electrodes, gains an electrical charge, and the level of the charge can be used to determine the quantity of ions of the electrolyte compound. The potentiometric sensor may be used to detect only one type of electrolyte. The potentiometric sensor may be disposed on a rigid or semi-rigid frame or substrate to function as a carrier for the sensor 42. In several embodiments, other electrochemical sensors are used such as amperometric or conductimetric to determine levels of concentrations of a particular attribute.

In several embodiments, the sensor 42 is a plurality of sensors. In one or more embodiments, the sensor 42 may be disposable as part of disposable portion 38a for each test of the attributes or replaced daily. In some embodiments, the non-disposable portion 38b may include the sensor 42. In one or more embodiments, the sensor 42 is color-changing to indicate the levels of the detected attributes, such as a colorimetric sensing device (e.g., ion-selective optodes, emulsified optodes, and solvent dyes). In one or more embodiments, the sensor 42 may be utilized to measure one or more attributes including, but not limited to: potassium, sodium, magnesium, pH, calcium, phosphate, glucose, and the like in waste fluid 32.

In one or more embodiments, the processor 44 analyzes and process data from the sensor 42 to determine electrolyte levels and/or PH levels and transmits that processed data to the transmitter 46. In several embodiments, the processor 44 determines peritoneal membrane characteristics. In some embodiments, based on the analyzed data, the processor 44 makes treatment recommendations such as an optimized treatment plan, which may include changing the composition of the dialysate 28. In some embodiments, the processor 44 analyzes hue or color data. In one or more embodiments, the processor 44 analyzes an electrical signal data. In several embodiments, the processor 44 determines electrolyte levels/concentrations or PH levels based on the analysis of processor 44. In some embodiments, the processor 44 compares threshold values of electrolyte concentration and/or PH levels to determine if the concentration and/or levels are normal, low, or high. In some embodiments the processor 44 filters the data transmitted by the sensor 42. In several embodiments, a plurality of instructions stored on a non-transitory computer readable medium may be executed by processor 44 to cause the processor 44 to carry out or implement in whole or in part the above-described operation of each of the described example embodiments. In one or more example embodiments, the processor 44 may include one or more of the microprocessors, any processor(s) that are part of the components of the system, and/or any combination thereof, and such a computer readable medium may be distributed among one or more components of the system. In several example embodiments, such a processor may execute the plurality of instructions in connection with the smart device 50. In several example embodiments, such a plurality of instructions may communicate directly with the one or more processors, and/or may interact with one or more operating systems, middleware, firmware, other applications, and/or any combination thereof, to cause the one or more processors to execute the instructions.

In one or more embodiments, the transmitter 46 uses Bluetooth® module to transmit the data. In some embodiments, the transmitter 46 includes a module for transmitting over the Internet, one or more local area networks, a low energy short-range wireless network such as Bluetooth®, one or more wide area networks, one or more cellular networks, one or more wireless networks, one or more voice networks, one or more data networks, one or more communication systems, and/or any combination thereof.

In some embodiments, the network 48 includes the Internet, one or more local area networks, a Bluetooth® low energy network, one or more wide area networks, one or more cellular networks, one or more wireless networks, one or more voice networks, one or more data networks, one or more communication systems, and/or any combination thereof. In some embodiments, multiple networks are used to transmit data.

In one or more embodiments, the smart device 50 is a cellular device and the graphical user interface 52 forms a portion of a mobile application. In some embodiments, the smart device 50 is a computer, personalized laptop, tablet, desktop computer, set of computers, cellular device (such as a smart phone) or the like. In some embodiments, the smart device 50 is remote from the sensor 42. In several embodiments, the smart device 50 is used by a patient. In other embodiments, the smart device 50 is used by a medical professional. In some embodiments, changes implemented by the smart device 50 (such as updated treatment plan) may be transmitted to database 54. In one or more embodiments, changes implemented by the smart device 50 and/or the processed data sent to the smart device 50 is also sent over network 48 to another computer (such as a patient computer).

In some embodiments, the graphical user interface 52 is or forms a portion of a patient-facing application that includes electronic health records. In some embodiments, the graphical user interface 52 displays the processed data. In one or more embodiments, the graphical user interface 52 displays indicators, which are graphical representations of the attributes. In some embodiments, the graphical user interface 52 displays a graph showing a change over time of the levels of the attributes. In some embodiments, the graphical user interface 52 displays changes in peritoneal membrane characteristics. In one or more embodiments, the graphical user interface 52 displays treatment recommendations such as an optimized treatment plan, which may include changing the composition of dialysate 28. In some embodiments, the graphical user interface 52 is updated on a regular basis such as daily or after each test, providing real-time data. In some embodiments, the graphical user interface 52 displays a recommendation to the patient to contact a medical professional or seek immediate medical attention. In one or more embodiments, the graphical user interface 52 is medical-professional facing. In some embodiments, the graphical user interface 52 displays the processed data to a medical professional. In one or more embodiments, the medical professional may select inputs on the graphical user interface 52 to update a patient's treatment plan such as changing the dialysate 28, recommending the patient come in for a check-up, or the like.

In some embodiments, the graphical user interface 52 displays automated messages. In other embodiments, the graphical user interface 52 displays messages from medical professionals to the patient regarding their PD session and/or treatment. In some embodiments, the sensor 42 may monitor and identify values that are outside of a predetermined acceptable band and a warning may be displayed via the graphical user interface 52. The graphical user interface 52 will display the measured attributes. In some embodiments, the measured attributes are tracked and graphed individually from each other. Additionally, a calculated relative transport status may be charted and displayed on the graphical user interface 52, in some embodiments. The graphical user interface 52, in some embodiments, may present historical data and/or display appropriate instructions in response to a value being outside an acceptable range. These instructions may be in regard to a PD treatment regimen or in regard to lifestyle adjustments such as modifications in diet, exercise, and sleep. In one or more embodiments, the graphical user interface 52 may also display articles and reading materials such as articles on electrolyte imbalance in response to the data being presented to the patient. In some embodiments, the graphical user interface 52 will receive input from the patient such as to schedule an appointment, including telehealth patient appointments. In some embodiments, the graphical user interface 52 displays a readout of the waste fluid, or spent dialysate, concentrations in mmol, mg/L, or compares the data to normal population serum values or target values input by the patient or the medical professional. In some embodiments, the graphical user interface 52 displays these values over time and provides an alert to the patient once a day on the data. That alert could be “good job, everything's normal,” include daily spikes or weekly trends, or the like.

In one or more embodiments, the database 54 is omitted. In some embodiments, the database 54 is a set of databases. In some embodiments, the database 54 is cloud-based storage. In some embodiments, the database 54 is accessible by only the medical professional, or only the patient. In other embodiments, the database 54 is accessibly by the medical professional and the patient to see the patient's data over time. In some embodiments, the database 54 stores the patient's data over time such as after each test. In some embodiments, the transmitter 46 automatically transmits the processed to the database 54 and then when a user (such as a patient or a medical professional) is ready to view the data on smart device 50, the smart device 50 pulls the data from the database 54 via network 48. In some embodiments, the database 54 is a portion of the electronic medical record of the patient. In some embodiment, the database 54 is part of the smart device 50. In some embodiments, the database 54 stores data from the PD session and other sessions to present a dashboard via the graphical user interface 52 for the patient of relevant data of the measured levels of the tested attributes, which may include one or more of glucose, pH, and electrolytes, including but not limited to sodium, calcium, magnesium, potassium, and phosphate. Further information, in several embodiments, may be stored in the database 54 such as session duration, photographs from the session, diet log, sleep log, and other variables that may impact or reflect the health of the patient. In one or more embodiments, the medical professionals may send, via network 48 and/or store messages in the database 54, such as messages with updated instructions for PD sessions. In some embodiments, the database 54 may send data to the smart device 50 and/or the graphical user interface 52 automatically as opposed to directly instructed from a medical professional. In one or more embodiments, the data collected and stored may trend a patient's transport status over time (in relation to a baseline value measured at the patient's first Peritoneal Equilibration Test). This transport status data, in some embodiments, may be used to make adjustments such as: increasing/decreasing dwell time per cycle; adding/removing a cycle/exchange; adding/subtracting a long or ambulatory dwell during the day; changing the prescription or concentration of the dialysate; changing the total time attached to the cycler in APD; changing the PD session times; and other important PD regimen adjustments.

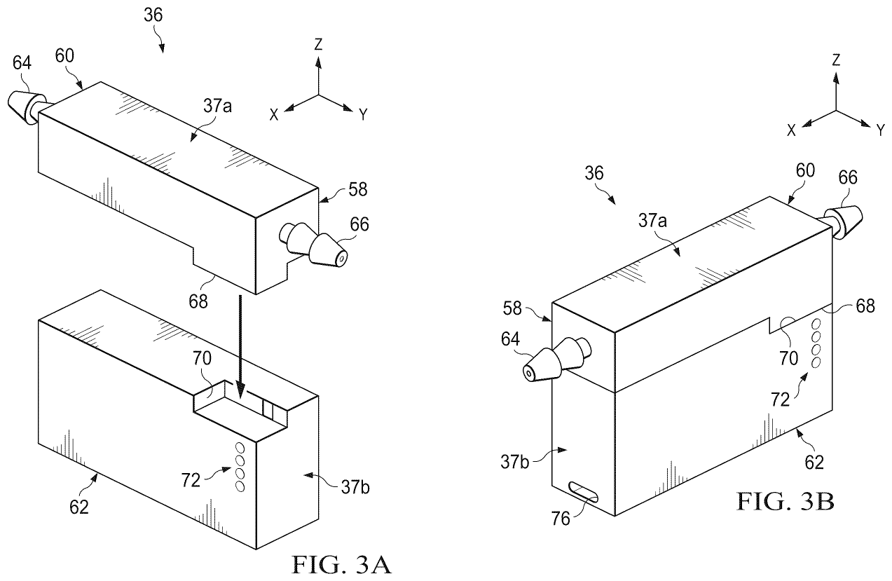

Referring to FIG. 3A, one embodiment of sensor assembly 36 is shown in more detail, where housing or frame 38 includes a first housing portion 60 and a second housing portion 62 shown separated from one another. The first housing portion 60 forms part of disposable portion 37a, and the second housing portion 62 forms part of non-disposable portion 37b. In the embodiment of FIG. 3A, waste fluid flow is disposed to pass through the disposable portion 37a where waste fluid can be detected by sensor 42 (see FIG. 2). As such, the first housing portion 60 includes a first tubing connector 64 and a second tubing connector 66. In one or more embodiments, the first tubing connector 64 and the second tubing connector 66. Tubing connectors 64, 66 may be axially aligned. The first housing portion 60 may also include a first connection mechanism 68 for engaging a second connection mechanism 70 on the second housing portion 62 in order to secure the first and second housing portions 60 and 62, respectively, to one another. The second housing portion 62 may also include indicators 72.

FIG. 3A shows first housing portion 60 separated from the second housing portion 62, while FIG. 3B, shows the first housing portion 60 seated on or otherwise engaging the second housing portion 62. FIG. 3B contains some of the components as shown in previous figures, which components are given the same reference numerals. The second housing portion 62 may also include a charging port 76.

In operation, with continuing reference to FIGS. 3A and 3B, in order to engage the first housing portion 60 with the second housing portion 62, the first connection mechanism 68 is inserted into the second connection mechanism 70 to couple the first housing portion 60 to the second housing portion 62. A charging cord (not shown) may be inserted into the charging port 76, and indicators 72 may indicate that the housing 58 is charging. The first and second tubing connectors 64 and 66, respectively, may be coupled to a drain line (not shown).

In several embodiments, the housing 58 may include different materials for the first housing portion 60 and the second housing portion 62.

In one or more embodiments, the first housing portion 60 having a sensor 42 (see FIG. 2) is replaced after each test to provide a new, unused sensor 42.

In one or more embodiments, the second housing portion 62 encloses a power supply 40, such as a battery, and a control unit, which may include a microprocessor such as processor 44, controller, or the like (see FIG. 2).

In some embodiments, the first tubing connector 64 and the second tubing connector 66 are supported by first housing portion 60 so as to be axially aligned. In one or more embodiments, the first tubing connector 64 and the second tubing connector 66 are push-lock connectors, a luer lock, or similar connectors that can couple the first housing portion 60 to a drain line. In some embodiments, the first tubing connector 64 and the second tubing connector 66 are hollow such that waste fluid may flow through the housing 58. In some embodiments, the first tubing connector 64 and the second tubing connector 66 are designed to be coupled to a drain line.

In one or more embodiments, the first connection mechanism 68 is a male connector that extends away from the remainder of the first housing portion 60. In some embodiments, the first connection mechanism 68 clips into, screws into, or otherwise fastens into the second connection mechanism 70 of the second housing portion 62. In some embodiments, the second connection mechanism 70 is a female connector that is indented within the second housing portion 62.

In some embodiments, the indicators 72 indicate when a sensor of the first housing portion 60 is detecting one or more attributes the waste fluid. In some embodiments, the indicators 72 include one or more lights. In some embodiments, the lights change color (green, yellow, red, and the like). In one or more embodiments, the indicators 72 indicate when the detection is complete. In some embodiments, the indicators 72 indicate an established connection such as an established Bluetooth® connection in order to transmit the test results (such as the processed data). In one or more embodiments, the indicators 72 indicate when a battery within housing 58 needs charging. In some embodiments, the indicators 72 indicate when a battery is being charged. In some embodiments, the indicators 72 are located directly under the first and second connection mechanisms, 68 and 70, respectively. Thus, while indicators 72 do not indicate the results of the measurements by sensor assembly 36, they can be utilized to indicate if a measurement has been taken or a measurement is complete or even that a measurement has begun or not begun and the progress of any measurement, thereby prompting a review of the results, as applicable.

In some embodiments, the charging port 76 is a USB-C, USB-A, USB-Micro, USB-Mini or the like. In some embodiments, the charging port 76 provides power to a battery housing within the second housing portion 62. In some embodiments, the charging port 76 also provides power to a sensor in the first housing portion 60 through the coupling of the first connection mechanism 68 with the second connection mechanism 70. In one or more embodiments, the charging port 76 is on the same side and/or plane as the first tubing connector 64.

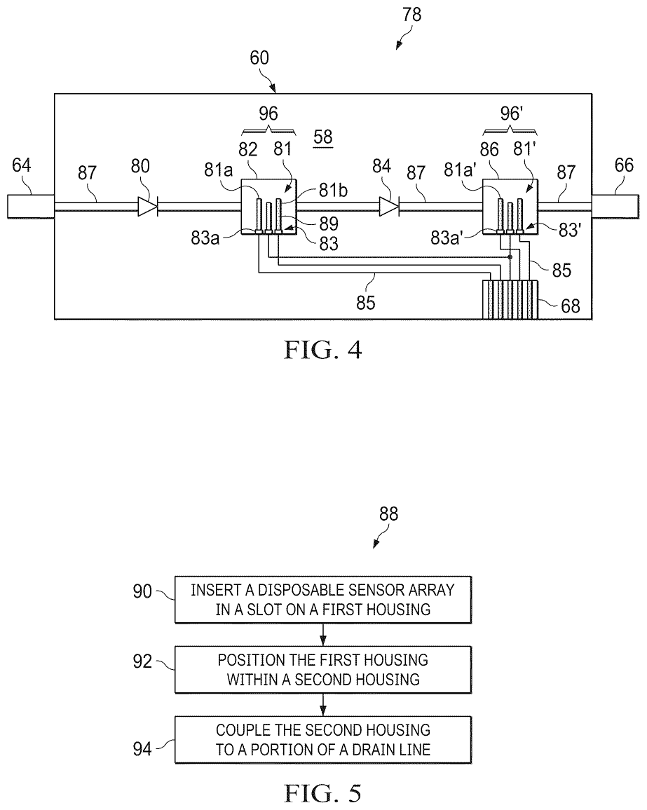

Referring to FIG. 4, with continuing reference to FIGS. 3A and 3B, in an embodiment, a cross-section of the first housing portion 60 of FIG. 3B is generally referred to by reference numeral 78, which contains some of the components as shown in previous figures, which components are given the same reference numerals, and includes the first housing portion 60 of the housing 58 and the first tubing connector 64 and the second tubing connector 66. The first housing portion 60 also includes at least a flow channel 87 extending between the first tubing connector 64 and the second tubing connector 66 with a first cell or sensing array 82 disposed along the flow channel 87. In some embodiments, the flow channel 87 may extend linearly through first housing portion 60 so as not to impede flow therealong. Flow channel 87 may be a conduit or tubing. A first valve 80 may be disposed along flow channel 87 between the first tubing connector 64 and the first cell 82. In one or more embodiments, first housing portion 60 may also include a second cell or sensing array 86 disposed along flow channel 87 between the first cell 82 and the second tubing connector 66. A second valve 84 may be disposed between the first cell 82 and the second cell 86. The first valve 80 is in fluid communication with and/or operably coupled with the first tubing connector 64 and the first cell 82. The first cell 82 is in fluid communication with and/or operably coupled to the first valve 80 and the second valve 84. The first cell 82 is also in electrical communication with processor 44 (not shown) and power supply 40 (not shown) via electrical lines 85. The second valve 84 is in fluid communication with and/or operably coupled to the first cell 82 and the second cell 86. The second cell 86 is in fluid communication with and/or operably coupled to the second valve 84 and the second tubing connector 66. The second cell 86 is also in electrical communication with processor 44 (not shown) and power supply 40 (not shown) via electrical lines 85.

The first cell 82 includes at least one microchannel 81 or at least one sensing material 89, along with at least one sensor 83 disposed adjacent the microchannel 81 or sensing material 89. In one or more embodiments, first cell 82 may include a plurality of microchannels 81 and/or a plurality of sensing materials 89. In some embodiments, sensing material 89 may be disposed within a microchannel 81, while in other embodiments, sensing material 89, if present, may be separate from microchannels 81. As used herein, sensing material 89 may be any material that absorbs a particular analyte or molecule or otherwise has a specific chemical reaction with an analyte, such as where a colorimetric sensing material that will change color in the presence of a particular analyte or molecule, which color change can then be detected by a sensor 83. In the illustrated embodiment, first cell 82 includes a plurality of microchannels 81, such as microchannel 81a. First cell 82 also illustrates a sensing material 81. Likewise, first cell 82 illustrates one or more sensors 83. In one or more embodiments, first cell 82 includes a plurality of sensors 83, such as sensor 83a disposed at or near an end of the one or more microchannels 81. In some embodiments, one sensor 83 is provided for each microchannel 81 or sensing material 89 present, thereby forming a cell or sensing array 96 of sensors 83 and microchannels 81 and/or sending materials 89. In any event, electrical lines 85 transmits data from sensors 83 to the processor 44 of the second housing portion 62 via the first connection mechanism 68.

Similarly, second cell or sensing array 86 may include one or more microchannels 81′, such as microchannel 81a′, and one or more sensors 83′, such as sensor 83a′, disposed at or near an end of the one or more microchannels 81′. Second cell 86 may alternatively or additionally include one or more sensing materials (not shown) as described above with respect to first cell 82. The electrical lines 85 transmits sensor data from the sensors 83′ to the processor 44 of the second housing portion 62 via the first connection mechanism 68. It should be noted that second cell 86 need not present in some embodiments. Moreover, second cell 86 may have microchannels 81′ that correspond with the microchannel 81 of first cell 82, thereby creating redundancy, or the microchannels 81′ of second cell 86 may differ in structure, number and function from microchannels 81 of first cell 82.

Although not necessary, in one or more embodiments, microchannels 81 may be parallel with one another. Moreover, although not limited to a particular diameter, in one or more embodiments, microchannels may be 300 micrometers or less in diameter. In one or more embodiments, microchannels may be 200 micrometers or less in diameter. In one or more embodiments, microchannels may be between 5 micrometers and 100 micrometers in diameter. Moreover, in one or more embodiments, the properties of any given microchannel, including diameter and length, as well as sensing material 89 if present, may be selected to “tune” the microchannel 81 to detect a particular analyte.

In operation, waste fluid (not shown) is directed from the first tubing connector 64 to the second tubing connector 66, so as to pass through the first valve 80 via flow channel 87, where a first portion of the waste fluid is exposed to the first cell 82. The first portion of the waste fluid enters at least the microchannel 81a of the one or more microchannels 81. In one or more embodiments, waste fluid may enter a microchannel 81 via capillary force, while in other embodiments, waste fluid may enter a microchannel 81 via absorption by a sensing material or absorbent disposed within the microchannel 81, such as is illustrated by microchannel 81b having a sensing material 89 disposed therein. When the portion of the waste fluid that is brought into a microchannel 81, such as microchannel 81a, reaches an opposite end of the microchannel 81, a sensor 83, such as sensor 83a, which may be disposed at the opposite end of the microchannel 81, detects one or more attributes of the portion of the waste fluid in the microchannel 81. Although shown as disposed at the end of a microchannel 81, a sensor 83 may be disposed anywhere along the length of a microchannel 81 so long as it performs the sensing function as described herein. The sensor 83 may determine a voltage or use another electrochemical process, as described herein, to detect one or more attributes of the first portion waste fluid. For example, at least one of the electrical lines 85 may provide a current to the sensor 83 from the power supply (not shown) in order test the potential difference (voltage). In other embodiments, the sensor 83 may detect a change in the color of a sensing material 89. In any event, The sensor 83 then transmits the data detected by the sensor 83 via the electrical lines 85 to the processor 44 in the second housing portion 62. The first remainder of the waste fluid, which was not tested by the first cell 82, passes through the flow channel 87 and the second valve 84 and a second portion of the waste fluid may then be exposed to the second cell 86, if present. The second portion of the waste fluid enters at least a microchannel 81′, such as microchannel 81a′ of the one or more microchannels 81′. When the second portion of the waste fluid that is brought into the microchannel 81a′ reaches an opposite end of the microchannel 81a′, a sensor 83′, such as sensor 83a′, detects one or more attributes of the second portion of the waste fluid in the microchannel 81a′. The sensor 83a′ may determine a voltage or use another electrochemical process detect one or more attributes of the second portion of the waste fluid. For example, at least one of the electrical lines 85 may provide a current to the sensor 83a′ from the power supply (not shown) in order test the potential difference (voltage). The sensor 83a′ then transmits the data detected by the sensor 83a′ via the electrical lines 85 to the processor 44 in the second housing portion 62. The second remainder of the waste fluid is then passed out of the first housing portion 60 via the second tubing connector 66.

In one or more embodiments, the first valve 80 is the same as the second valve 84. In other embodiments, the first valve 80 differs from the second valve 84. In some embodiments, only one valve is present. In other embodiments, multiple valves are included in the first housing portion 60 of the housing 58. In some embodiments, the first valve 80 and/or the second valve 84 circumvents the majority of the waste fluid from being tested by the first cell 82 and/or the second cell 86, which causes the majority of the waste fluid to exit the housing 58. In some embodiments, the first valve 80 and the second valve 84 are anti-reflux valves.

In some embodiments, one test cell (such as the first cell 82 or the second cell 86) is omitted. In several embodiments, the first cell 82 and/or the second cell 86 only detect for one substance such as, but not limited to, glucose, sodium, or potassium. In one or more embodiments, the first cell 82 and/or the second cell 86 includes different microchannels 81 and 81′, respectively, and/or different sensors 83 and 83′ in a sensing array 96, to detect a different plurality of attributes (of the waste fluid). In several embodiments, the plurality of microchannels 81 may be parallel to one another. In some embodiments, the plurality of microchannels 81 are different lengths. In several embodiments, the first cell 82 and/or the second cell 86 includes the same type of microchannels 81 and 81′ and/or the same types of sensors 83 and 83′ for the sensing arrays 96, 96′ respectively, to detect the same plurality of attributes (of the waste fluid) in the first cell 82 and the second cell 86. In some embodiments, one or more microchannels 81, 81′ may be a microfluidic channel made of cellulose, nylon, or another membrane material with capillary activity (capillary force) that may draw up fluid (from drain tube). In some embodiments, the plurality of microchannels 81 or the plurality of microchannels 81′ is replaced with only a single microchannel. In some embodiments, the first cell 82 and/or the second cell 86 includes one main channel and a plurality of diverging microchannels. In some embodiments, the plurality of microchannels 81 each have a sensor 83, such as sensor 83a, specific for one analyte: such as glucose, sodium, potassium, calcium, phosphate, pH, and magnesium. In some embodiments, the plurality of microchannels 81′ each have a sensor 83, such as sensor 83a, specific for one analyte: such as glucose, sodium, potassium, calcium, phosphate, pH, and magnesium. In some embodiments, one or more microchannels 81, such as microchannel 81a, and one sensor 83, such as sensor 83a, measure creatinine, BUN, H, bicarbonate, and/or lactate. In several embodiments, one microchannel of the plurality of microchannels 81a′ such as microchannel 81a′ and one sensor 83′ accompanying sensor 83a′ are used to measure creatinine, BUN, H, bicarbonate, and/or lactate. In other embodiments, the first cell 82 and/or the second cell 86 test for a plurality of substances such as, but not limited to, glucose, sodium, or potassium. In some embodiments, the first cell 82 includes a first set of microchannels and respective sensors, and the second cell includes a second set of microchannels and respective sensors. In some embodiments, the first cell 82 tests for one or more substances different than the substances tested by the second cell 86. In some embodiments, the first cell 82 and/or the second cell 86 are coupled to a power source such as a battery (located in the second housing portion 62), and the power source applies a current on the first cell 82 and/or the second cell 86. In some embodiments, data from the first cell 82 and the second cell 86 are transmitted to a processor located in the second housing portion 62 of the housing 58, which is processed and transmitted to a computer and/or an application on a smart device.

With reference to FIG. 5, a method 88 for monitoring peritoneal dialysis (PD) system utilizing a sensor assembly is described, according to one or more embodiments. Method 88 is illustrated as a set of operations or blocks 90 through 94. Not all of the illustrated blocks 90 through 92 may be performed in all embodiments of method 88. One or more blocks that are not expressly illustrated in FIG. 5 may be included before, after, in between, or as part of the blocks 90 through 94. In one or more embodiments, the blocks in method 88 are performed as part of setting up a sensor assembly of a PD system, such as the sensor assembly 18 of the PD system 10 in FIG. 1 or any other embodiment disclosed herein. In some embodiments, the blocks in method 88 are performed as part of setting up a sensor assembly for a PD system, such as the sensor assembly 18, or any of the other embodiments described herein.

In an example embodiment, the method 88 includes: inserting a disposable sensing array in a slot on a first housing at a block 90 where the sensing array is tuned to detect one or more analytes in waste fluid; positioning the first housing within a second housing at a block 92; and coupling the second housing to a portion of a waste fluid drain line at a block 94. It should be understood that in some embodiments of block 90, the disposable sensor array is first selected to identify one or more desired analytes. Selecting the a sensor array comprises tuning the sensor array by incorporating one or more microchannels and/or colorimetric sensors disposed to react or interact with the desired analytes. This may involve incorporating microchannels of size, dimension, length, shape and/or sensing material that are best disposed to absorb the desired analyte.

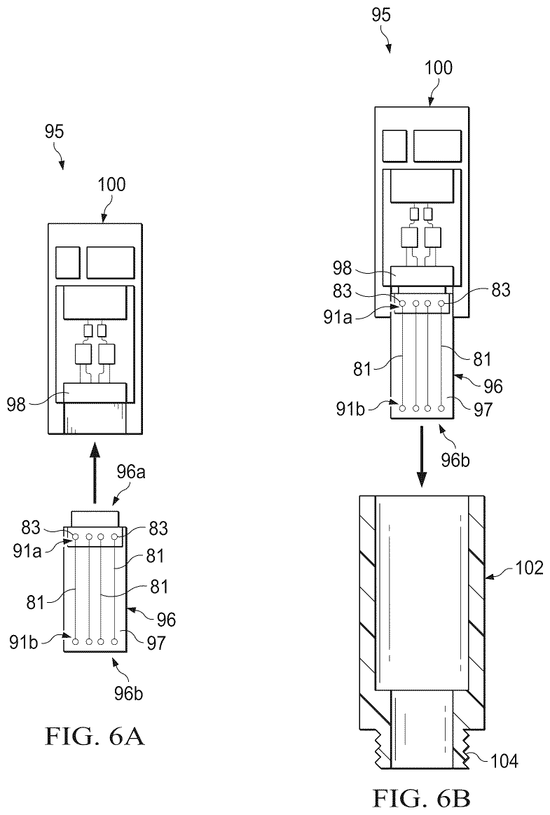

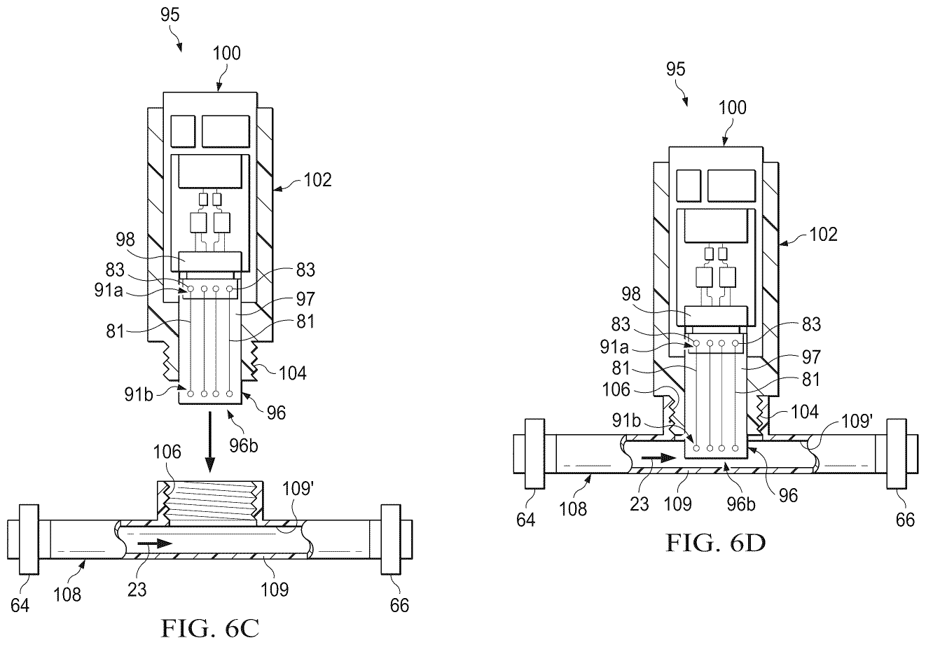

With reference to FIGS. 6A-6D, another embodiment of sensor assembly 18 is shown in more detail and is generally referred to by reference numeral 95. In this embodiment, sensor assembly 95 includes a disposable portion 99a and a non-disposable portion 99b. Unlike the embodiments of FIGS. 3A, 3B and 4, in the embodiment of sensor assembly 95, waste fluid does not pass through the disposable portion 99a, but rather, the disposable portion 99a is a sensing array 96 that can be detached from the non-disposable portion 99b and discarded. Moreover, in this embodiment, the disposable portion 99a is positioned in the waste flow path 23 of a drain line assembly 108. With continuing reference to FIG. 5, in an embodiment, FIGS. 6A-6D illustrates method 88, in one embodiment, by showing a progression of assembling a sensor assembly 95. Sensor assembly 95 includes a sensing array 96 having one or more microchannels 81, each microchannel 81 having a first end 91a and a second end 91b. Microchannels 81 may be as described above with reference to the preceding Figures. In one or more embodiments, a sensor 83 id disposed adjacent the first end 91a of a microchannel 81. In the illustrated embodiments of FIGS. 6A-6D, a plurality of microchannels 81 are illustrated along with a plurality of sensors 83. Sensing array 96 is disposed in a slot 98 formed in a housing 100. The housing 100 supports one or more of the electrical components described above in FIG. 2. The housing 100 may then be supported in a outer housing 102 that has a first connector 104 disposed to be releasably engaged with a second connector 106 of a drain line assembly 108. Drain line assembly 108 includes a peritoneal dialysis waste fluid vessel 109 in the form of a conduit, and further includes a first tubing connector 64 and a second tubing connector 66 in fluid communication with the conduit 109.

In some embodiments, with continuing reference to FIGS. 5-6D, the block 90 includes inserting a first end 96a of sensing array 96 into the slot 98. In one or more embodiments, the block 94 includes coupling the outer housing 102 to a portion of the drain line assembly 108. The sensing array 96 may include microchannels 81 and sensors 83. In some embodiments, the sensing array 96 is disposable. In one or more embodiments, the sensing array 96 is one or more test cells such as the first cell 82 and/or the second cell 86. In some embodiments, the sensing array 96 includes a carrier 97 made of paper, metal, plastic, a frame or other substrate, which may, in some embodiments, be semi-rigid or rigid to support the microchannel 81 and/or sensors 83. In some embodiments, the sensing array 96 includes sensors 83 printed on carrier 97. In some embodiments, sensors 83 may be disposed in a row on a carrier 97. In some embodiments, the carrier 97 may have dimension of approximately 1″ in width and 4.5″ in length. In some embodiments, smaller microchannels 81 may branch off from a main microchannel 81 to direct fluid to each of these sensors 83. In some embodiments, other dimensions and geometric arrangements may be used. Where carrier 97 is paper, the paper may have a thickness of 9-20 micrometers in some embodiments, and may have a thickness of 160-390 micrometers in some embodiments. The thickness of the carrier 97 may vary depending the analyte sample needed for each sensor 83 and based on the capillary force needed to draw a sufficient amount of sample fluid from the utilizing microchannels 81. In some embodiments, carrier 97 may be porous, absorbing sample fluid that can then be drawn into microchannels 81 of the porous carrier 97. In some embodiments, the sensing array 96 includes colorimetric sensors, which may all start at the same huc/color e.g., blue hue, or at different hues/colors. In some embodiments the sensing array 96 is inserted into a slot 98 within the housing 100 in the block 90. In some embodiments, a second end 96b of sensing array 96 extends past the perimeter of the housing 100, for example, as shown in FIG. 6A. In one or more embodiments, the second end 91b of a microchannel 81 is adjacent the second end 96b of a sensing array 96.

In some embodiments, the block 92 includes positioning the housing 100 within an outer housing 102. In one or more embodiments, the second end 96b of sensing array 96 that extends past the housing 100 is inserted into the outer housing 102, for example, as shown in FIG. 6B. In some embodiments, the second end 96b of sensing array 96 that extends past the housing 100 also extends past the outer housing 102. In some embodiments, the outer housing 102 is omitted. In some embodiments, the block 92 includes coupling a first housing portion of the housing 100 to a second housing portion of the housing 100.

In some embodiments, the block 94 includes inserting the sensing array 96 into the drain line assembly 108, for example as shown in FIG. 6C. In some embodiments, the sensing array 96 extends within the first connector 104.

In some embodiments, the block 94 includes coupling the outer housing 102 to a portion of a drain line assembly 108, for example, as shown in FIG. 6D. In some embodiments, at the block 94, the first and second connectors 104, 106 are used to couple the outer housing 102 to a portion of the drain line assembly 108. housing 100drain line assembly 108 In some embodiments, the first connector 104 is a single connector. In other embodiments, the first connector 104 is a plurality of connectors. In some embodiments, the second end 96b of sensing array 96 extends into the waste flow path 23 defined within conduit 109 so that the second end 91b of microchannels 81 can be exposed to waste fluid passing therethrough. In some embodiments, the first connector 104 is any one or more of: an in-line connector that couples the first connector 104 to the drain line assembly 108 such as a luer lock, latch, adhesive, stitching, screw, an attachment to drain line assembly 108, and like. In some embodiments, the second connector 106 is omitted, and the first connector 104 directly couple to the drain line assembly 108 instead (as described herein). In some embodiments, the second connector 106 is a single connector. In other embodiments, the second connector 106 is a plurality of connectors. In some embodiments, the second connector 106 is any one or more of: an in-line connector that couples the first connector 104 to the drain line assembly 108 such as a luer lock, latch, adhesive, stitching, screw, attachment to drain line assembly 108, and like. In some embodiments, the second connector 106 forms a portion of the drain line assembly 108, and in particular, the conduit 109. In one or more embodiments, the second end 96b of sensing array 96 extends past the first connector 104 and the second connector 106 (which are coupled together) and is disposed within the drain line assembly 108, for example, as shown in FIG. 6D. In some embodiments, the sensing array 96 is in direct contact with an interior sidewall 109′ of conduit 109 of the drain line assembly 108 to ensure that the waste fluid contacts the sensing array 96. In some embodiments, the drain line assembly 108 is coupled to a PD catheter and/or a drainage bag (see FIG. 1). In some embodiments, the housing 100 is coupled to a portion of the drain line assembly 108 at the block 94. In some embodiments, both the housing 100 and the outer housing 102 are coupled to the drain line at the block 94.

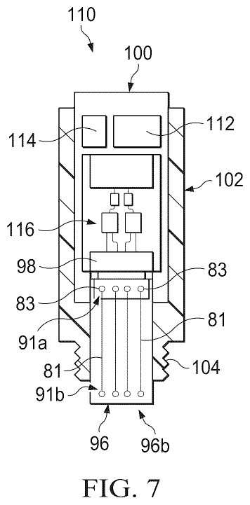

Referring to FIG. 7, with continuing reference to FIGS. 6A-D, in an embodiment, a sensor assembly is generally referred to by reference numeral 110 and contains some of the components as shown in previous figures, which components are given the same reference numerals. The sensor assembly 110 includes the housing 100 that includes a Bluetooth® chip 112, a power source 114, a microprocessor 116, and the slot 98. The sensor assembly 110 also includes the outer housing 102 that is coupled to at least a portion of the housing 100. The outer housing 102 includes the first connector 104. The sensor assembly 110 also includes the sensing array 96. The power source 114 is in communication with and/or operably coupled to the Bluetooth® chip 112, the microprocessor 116, and/or the sensing array 96. The microprocessor 116 is in communication with and/or operably coupled to the battery 114, the Bluetooth® chip 112, and/or the sensing array 96. The Bluetooth® chip 112 is in communication with and/or operably coupled to the battery 114, the microprocessor 116, and/or the sensing array 96. The sensing array 96 is in communication with and/or operably coupled to microprocessor 116.

In some embodiments, the housing 100 is disposed within at least a portion of the outer housing 102. In some embodiments, the outer housing 102 surrounds the housing 100. In other embodiments, the housing 100 is only coupled to the outer housing 102 and is not disposed within the outer housing 102.

In some embodiments, the Bluetooth® chip 112 is replaced with an alternative transmitter. The alternative transmitter may use Wi-Fi, cellular network, or the like.

In some embodiments, the power source 114 is chargeable via a charging port and a charging cord. In other embodiments, the power 114 is one or more batteries.

In some embodiments, the microprocessor 116 includes one or more microprocessors. In some embodiments, electronic circuitry couples one or more of: the microprocessor 116, the sensing array 96, the power source 114, and/or Bluetooth® chip 112.

In some embodiments, the slot 98 is a receiving slot for the sensing array 96. In some embodiments, the slot 98 is an indented portion of the housing 100. In some embodiments, there are a plurality of first slots to accommodate a plurality of sensor arrays. In some embodiments, the slot 98 accommodates the sensing array 96 having electrical connection to the microprocessor 116 and/or the power source 114. In some embodiments, the slot 98 is disposed to receive and secure the first end 96a of sensing array 96.

In several embodiments, the outer housing 102 is omitted. In some embodiments, the outer housing 102 is instead a second portion of the housing 100.

In some embodiments, the outer housing 102 includes a plurality of second slots to accommodate a plurality of sensor arrays, such as sensing array 96. In one or more embodiments, second slot is the same width as the slot 98 to accommodate the sensing array 96. In some embodiments, the second slot extends within the first connector 104 and the outer housing 102. In one or more embodiments, the slot 98 is also within the outer housing 102.

In several embodiments, the first connector 104 is a male or a female connector that slides-into the opposite type of connector. In other embodiments, the first connector 104 is threaded. In other embodiments the first connector 104 is a plurality of connectors. In some embodiments, the first connector 104 is a first connector that is coupled to a first portion of a drain line and a second connector that is coupled to a second portion of a drain line (as described herein).

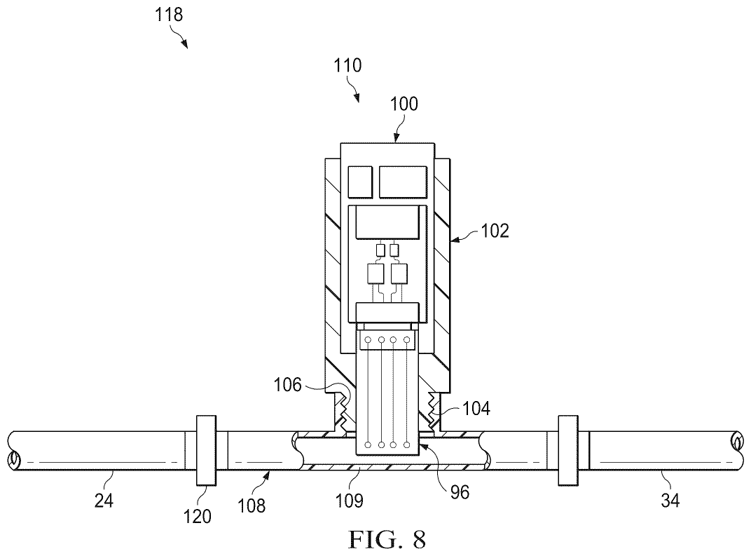

Referring to FIG. 8, with continuing reference to FIGS. 6-7, in an embodiment, a a portion of a PD system 118 is shown with having a sensor assembly 110 and contains some of the components as shown in previous figures, which components are given the same reference numerals. The sensor assembly 110 coupled to the drain line assembly 108 via first connector 104 and second connector 106. The drain line assembly 108 includes one or more line tubing connectors 120 and a conduit 109 of the drain line assembly 108. Connectors 120 allow drain line assembly 108 to be removable inserted along a waste line 24 such that drain line assembly 108, like sensing array 96, is disposable. However, drain line assembly 108 permits sensor assembly 118 to be readily installed along a drain line of a PD system.

In one or more embodiments, the line tubing connectors 120 and the disposable portion 122 are instead disposed within the housing 100 and/or the outer housing 102 as described above with respect to FIGS. 3A, 3B and 4.

In some embodiments, the line tubing connectors 120 are the first connector 104 and the second connector 106. In some embodiments, the line tubing connectors 120 couple the disposable portion 122 to the rest of the drain line assembly 108. In some embodiments, the line tubing connectors 120 are also disposable. In some embodiments, the line tubing connectors 120 couple the disposable portion 122 to a catheter and/or drainage bag. In some embodiments, the line tubing connectors 120 are plastic and are a valve.

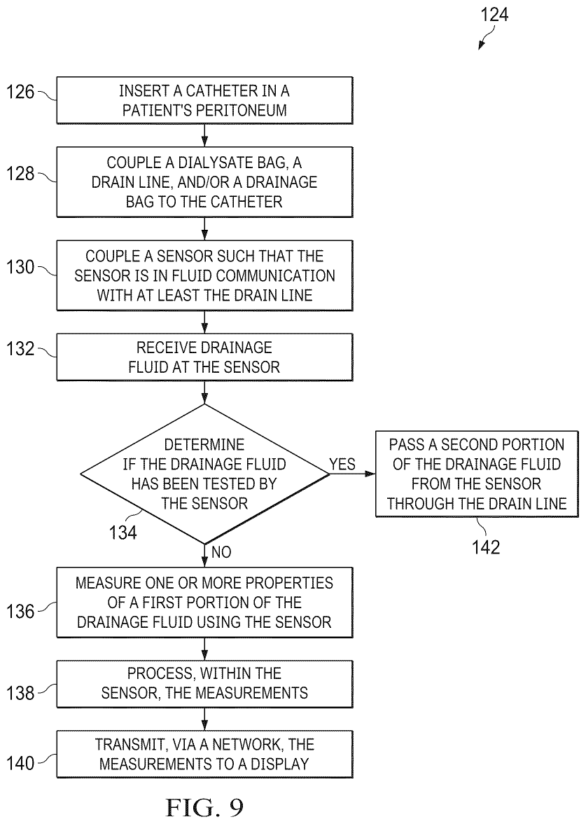

With reference to FIG. 9, a method 124 for attribute-sensing PD, according to one or more embodiments. Method 124 is illustrated as a set of operations or blocks 126 through 142. Not all of the illustrated blocks 126 through 142 may be performed in all embodiments of method 124. One or more blocks that are not expressly illustrated in FIG. 9 may be included before, after, in between, or as part of the blocks 126 through 142. In some embodiments, one or more of the blocks 126 through 142 may be implemented, at least in part, by a controller which may include a processor, such as processor 44 or microprocessor 116, in the form of executable code stored on non-transitory, tangible, machine-readable media that when run by one or more processors may cause the one or more processors to perform one or more of the processes. In one or more embodiments, the blocks in method 124 are performed within a PD system, such as PD system 10, sensor assembly 110, or any of the other embodiments described herein.

The method 124 includes inserting a catheter in a patient's peritoneum at a block 126, coupling a dialysate bag, a drain line, and/or a drainage bag to the catheter at a block 128; coupling a sensor such that the sensor is in fluid communication with at least a drain line and/or a drainage bag at a block 130; receiving drainage fluid at the sensor at a block 132; and determining if the drainage fluid had been tested by the sensor, at a block 134. If not, then proceed to a block 136, the block 136 provides: measuring one or more properties of a first portion of the drainage fluid, using the sensor, at a block 136; processing, within the sensor, the measurements at a block 138; transmitting, via a network, the measurements to a display at a block 140. If yes, then, proceed to a block 142, the block 142 provides passing a second portion of the drainage fluid from the sensor through the drain line.

In some embodiments, the block 126 occurs at an earlier point in time than blocks 123-142. In some embodiments, the catheter is catheter 14.

In one or more embodiments, coupling a dialysate bag, a drain line, and/or a drainage bag occurs at different points in time. In some embodiments, only a drain line is used and not a drainage bag. In some embodiments, coupling a dialysate bag occurs earlier than coupling a drain line and/or a drainage bag. The drain line, in some embodiments, may be drainage line 34 and may include waste line 24. In other embodiments, the drain line may be drain line assembly 108 and may include the disposable portion 122. In some embodiments, the dialysate bag is dialysate bag 26. In some embodiments, the drainage bag is the waste receptacle 30. In some embodiments, the dialysate bag and the drainage bag are coupled to the catheter via the drain line.

In some embodiments, the block 130 occurs before or simultaneously to block 128. In some embodiments, the sensor is the sensor assembly 18, sensor 42, first cell 82, second cell 86, sensing array 96, sensor assembly 110, or the like.

In some embodiments, at the block 132, the sensor receives drainage fluid from the drain line because the sensor extends within a portion of the drain line. In some embodiments, at the block 132, the sensor forms a portion of the drain line. In other embodiments, at the block 132, the sensor receives fluid from the waste receptacle because the sensor is positioned within the waste receptacle. In some embodiments, at the block 132, the sensor receives waste fluid from openings on the sensor and the waste fluid passes through microchannels to a sensing portion of the sensor disposed at the end of the microchannel. In some embodiments, the sensor produces an electrical signal or a color-change of the sensor occurs when the drainage fluid is received.

In one or more embodiments, the block 134 is automatic. In some embodiments, the block 134 is omitted. In some embodiments, a controller or processor determines if a sensor has tested the drainage fluid. In other embodiments, once the sensor is used, then it no longer works, which directs the fluid to pass through the drain line rather than being tested. In some embodiments, the sensor, such as an ISE, reads a value “0” showing that the sensor has not yet come in contact with the waste fluid. In some embodiments, the sensor, such as an ISO, has yet to change color showing that the sensor has not yet come in contact with the waste fluid.

In some embodiments, the block 136 occurs simultaneously to the block 142. In one or more embodiments, the block 136 precedes the block 142. In some embodiments, the attributes being measured change color in the sensing array 96 or produces another qualitative value. In some embodiments, the attributes produce a quantitative value at the block 136, such as an electrical signal.

In one or more embodiments, the blocks 138 and 140 occur at the same time as the block 142. In some embodiments, the measurements are processed by a microprocessor as described herein within the sensor. In some embodiments, the measurements undergo a first processing within the sensor and a second processing outside of the sensor (i.e., within a computer after the block 140). In some embodiments, the measurements are processed via a filter.

In some embodiments, the network at the block 140 is a network described herein such as a Wi-Fi, cellular, Bluetooth®, network 48, and the like. In some embodiments, the transmission occurs over a wired connection. In some embodiments, the display is the graphical user interface 52. In some embodiments, the display is remote to the sensor. In other embodiments, the display is instead integrated into the sensor. In some embodiments, the integrated display reads out a graphical indicator or number to the patient. For example, the sensor may include a hue measuring electronic device (described below), a pH sensor (described below), or an alternative reader that reads the sensor and presents the graphical indicator or number to the patient.

In one or more embodiments, the block 142 passes a second portion or the remainder of the drainage fluid from the sensor through the drain line into a drainage bag or toilet. In some embodiments, the block 142 passes a second portion or the remainder of the drainage fluid (or waste fluid) through the drain line to a drainage bag.

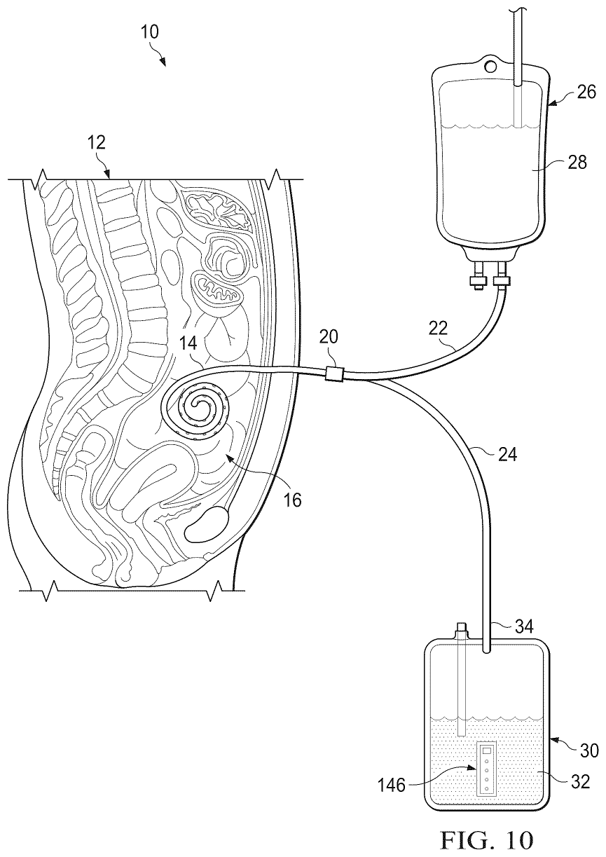

Referring to FIG. 10, one embodiment of the PD system 10 is shown in more detail where sensor assembly 18 is shown in more detail and is generally referred to by reference numeral 146. Notably, FIG. 10 illustrates an embodiment of a PD system 10 where the sensor assembly 146 is disposed on or in waste receptacle 30 as opposed to being disposed along waste line 24 as described above with respect to FIG. 1. The system of FIG. 1 allows only a portion of the sensor assembly 18, namely the cell or sensing array as described above, to be removed and replaced without the need to discard the entire sensor assembly and without the need to replace waste receptacle 30 prior to a new measurement. In other words, waste receptacle 30 can continue to be used over a period of time while multiple measurements over that period of time using sensor assembly 18 are performed. In contrast, in some embodiments, each time a measurement is desired using the sensor assembly 146 of FIG. 10, the entire sensor assembly 146 and waste receptacle 30 may need to be replaced. In any event, in the embodiments of FIG. 10, sensor assembly 146 is exposed to waste fluid 32 passing from catheter 14 and into waste receptacle 30. The catheter 14 is in fluid communication with dialysate bag 26, which contains dialysate 28 and is in fluid communication with a waste receptacle 30, which collects waste fluid 32. The catheter 14 is coupled to the connector 20, which couples the supply line 22 and the waste line 24 to the catheter 14. The supply line 22 is operationally coupled to dialysate bag 26, and the waste line 24 is operationally coupled to the waste receptacle 30. Ad described above, in this embodiment, rather than positioning sensor assembly 146 along a waste line 24, the sensor assembly 146 is carried by waste receptacle 30. In some embodiments, sensor assembly 146 may include the sensing array 96, as well as a power source 114, the Bluetooth® chip 112, the microprocessor 116 as described above operating in conjunction with the sensing array 96.

In several embodiments, the waste line 24 includes one or more portions of drain line. In some embodiments, the waste line 24 includes a luer lock.

In one or more embodiments, the sensor assembly 146 is attached in an interior region of the waste receptacle 30. In some embodiments, the sensor assembly 146 is visible, even though the sensor assembly 146 is disposed within the waste receptacle 30. In several embodiments, the waste receptacle 30 is made of plastic, film, or other clear substance. In some embodiments, the sensor assembly 146 is made integral to the waste receptacle 30. In other embodiments, at least a portion of sensor assembly 146 is attachable and separate to the waste receptacle 30. In several embodiments, the sensor assembly 146 and/or the waste receptacle 30 is disposable.

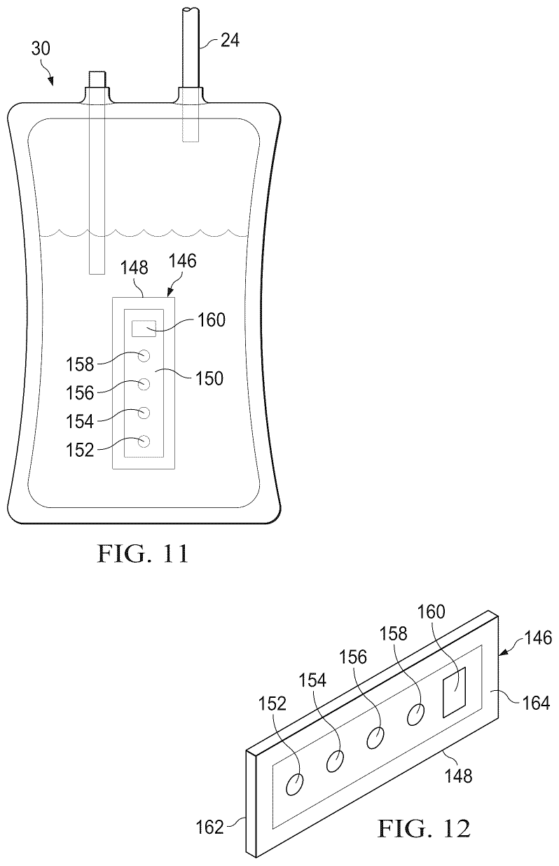

Referring now to FIG. 11, the sensor assembly 146 supported on the waste receptacle 30 is shown in more detail. The sensor assembly 146 may include a frame or housing 148 to provide support for a central carrier 150. The central carrier 150 may include one or more of a first sensor 152, a second sensor 154, a third sensor 156, a fourth sensor 158, and a fifth sensor 160. In some embodiments, one or more of the first sensor 152, second sensor 154, third sensor 156, fourth sensor 158, and fifth sensor 160 may be sensors 83 as described above.

In some embodiments, the housing or frame 148 is composed of a rigid or a semi-rigid material. In one or more embodiments, the housing or frame 148 surrounds the central carrier 150. In other embodiments, the frame 148 supports the central carrier 150 and is beneath the central carrier 150, as well as, surrounding the central carrier 150. In yet other embodiments, the frame 148 is omitted. In some embodiments, the frame 148 is instead adhesive that surrounds the central carrier 150 that couples the central carrier 150 to the waste receptacle 30.

In one or more embodiments, the central carrier 150 may be omitted. In some embodiments, the central carrier 150 and/or the frame 148 may be another shape besides a rectangle. In some embodiments, the central carrier 150 is a paper carrier such as paper or Nylon-based paper. In several embodiments, the central carrier 150 is replaceable after each use. In some embodiments, the central carrier 150 is inserted into the frame 148. In some embodiments, the central carrier 150 may include only one sensor. In other embodiments, the central carrier 150 may include a plurality of sensors (such as, but not limited to: the first sensor 152, the second sensor 154, and the like). In one or more embodiments, the central carrier 150 is attached to the waste receptacle 30 via adhesive, stitching, manufacturing, or the like. In several embodiments, the central carrier 150 is disposed at a bottom of the waste receptacle 30, opposite to the waste line 24.

In some embodiments, one or more of: the first sensor 152, the second sensor 154, the third sensor 156, the fourth sensor 158, and/or the fifth sensor 160 are different types of sensors. Different types of sensors may include colorimetric sensors, electrochemical sensors, metal-based sensors, hue measuring device, pH sensors, and the like. In some embodiments, the different types of sensors may include ISE, as ion-selective optodes (ISO) sensors, emulsified optodes, solvent dyes, or the like. In one embodiment, the first sensor 152, the second sensor 154, the third sensor 156, and the fourth sensor 158 are colorimetric sensors, but the fifth sensor 160 is either a pH sensor or a hue sensor. In some embodiments, all of the first sensor 152, the second sensor 154, the third sensor 156, the fourth sensor 158, and/or the fifth sensor 160 are the same type of sensor and/or same shape. In some embodiments, all of the first sensor 152, the second sensor 154, the third sensor 156, the fourth sensor 158, and/or the fifth sensor 160 are the same type of sensor but measure a different type of attribute. In one or more embodiments, one or more of: the first sensor 152, the second sensor 154, the third sensor 156, the fourth sensor 158, and/or the fifth sensor 160 are one or more colorimetric or color changing sensors such as ISO put on the central carrier 150, such as paper e.g., Whatman® paper or Nylon-based paper. In some embodiments, a portion of the first sensor 152, the second sensor 154, the third sensor 156, the fourth sensor 158, and/or the fifth sensor 160 are an electrochemical sensor and while another portion of one or more of: the first sensor 152, the second sensor 154, the third sensor 156, the fourth sensor 158, and/or the fifth sensor 160 are a colorimetric sensor. In some embodiments, when an ISO sensor is used, the hue measuring device is also one of the first sensor 152, the second sensor 154, the third sensor 156, the fourth sensor 158, or the fifth sensor 160 are. In some embodiments, the first sensor 152, the second sensor 154, the third sensor 156, the fourth sensor 158, and the fifth sensor 160 may be disposed in any orientation on the waste receptacle 30 so long as the first sensor 152, the second sensor 154, the third sensor 156, the fourth sensor 158, and the fifth sensor 160 are visible from outside of the waste receptacle 30 and the first sensor 152, the second sensor 154, the third sensor 156, the fourth sensor 158, and the fifth sensor 160 are in fluid communication with the waste fluid 32.

Referring to FIG. 12, one embodiment of the sensor assembly 146 is shown in more detail and includes a back side 162 and a front 164. The front 164 includes the first sensor 152, the second sensor 154, the third sensor 156, the fourth sensor 158, or the fifth sensor 160. The back side 162 is where the waste fluid 32 in the waste receptacle 30 interfaces with the first sensor 152, the second sensor 154, the third sensor 156, the fourth sensor 158, or the fifth sensor 160 disposed on the central carrier 150, and the front side 164 may be where the color change is visible from outside of the waste receptacle. In some embodiments, the sensor assembly 146 also includes microfluidic channels or microchannels or sensing material as described above that allow a portion of the waste fluid 32 to travel from the back side 162 to the front side 164, where the sensors (such as the first sensor 152, the second sensor 154, and the like) are disposed, in order for the portion of the waste fluid 32 to be measured by the sensors.

In operation, with continuing reference to FIGS. 11 and 12, the dialysate 28 flows from the dialysate bag 26 through the supply line 22 through the catheter 14 into the patient's peritoneum 16. When the dialysate 28 is in the body of the patient, the solution absorbs waste and extra fluid from the body. Then, the waste fluid 32 is drained using the catheter 14 and waste line 24. As the waste fluid 32 is drained, it collects in the waste receptacle 30. The sensor assembly 146, which is coupled to the waste receptacle 30, may detect concentration levels for a variety of attributes such as glucose, sodium, calcium, magnesium, phosphate, and/or the like, in real-time, during the PD treatment. In some embodiments, one or more of: the first sensor 152, the second sensor 154, the third sensor 156, the fourth sensor 158, or the fifth sensor 160 change color depending on the measured concentration of the attribute detected by the respective sensor.

In some embodiments, the frame 148 may be moved or positioned from outside of the waste receptacle 30 for visibility or to position the sensors (such as the first sensor 152, the second sensor 154, the third sensor 156, the fourth sensor 158, and the fifth sensor 160) in an appropriate position for detection.

In one or more embodiments, the sensor assembly 146 measures various attributes of the waste fluid 32. In some embodiments, the sensor assembly 146 measures electrolytes and/or the pH of the waste fluid 32. In several embodiments, the attributes measured by the sensor assembly 146 includes potassium, glucose, magnesium, sodium, pH, calcium, phosphate, and the like. In several embodiments, each sensor of the first sensor 152, the second sensor 154, the third sensor 156, the fourth sensor 158, and the fifth sensor 160 detects a different attribute. In some embodiments, the sensor assembly 146 measures electrolyte concentration and/or glucose levels. In some embodiments, the sensor assembly 146 may measure sodium ion concentration of dialysis effluent (100-200 mmol/L, 1 mmol resolution), potassium ion concentration of dialysis effluent (0-10 mmol/L, 1 mmol/L), calcium ion concentration of dialysis effluent (0-10 mmol/L, 1 mmol/L), phosphate ion concentration of dialysis effluent (0-20 mmol/L, 1 mmol/L), and glucose concentration of dialysis effluent (0-250 mmol/L, 1 mmol/L). Other attributes of the waste solution may be detected in other embodiments. In some embodiments, sodium measurements may establish a baseline for measurement of potassium in the case of cross reactivity of sodium with other ionophores. In some embodiments, incorporation of other readings may be used including urea, blood urea nitrogen (BUN), bicarbonate, lactate, and other diagnostic features. For example, creatinine and BUN or other attributes (or analytes) of interest may be detected for the purpose of replicating a Peritoneal Equilibration Test and/or for the measurement of Kt/V, a common metric for measuring the filtration rate of a patient's peritoneum. In some embodiments, a different sensing modality is needed within sensor assembly 146 that is not an ISO or ISE, such as but not limited to enzyme-linked immunoassay (ELISA), Immunofluorescence, Western Blot, or Antibody testing-such as for testing creatinine, BUN, lactate, urea, cells, or other proteins. In some embodiments, bicarbonate and lactate are attributes of interest due their potential in assessing a patient's acid-base status. In some embodiments, sensor assembly 146 measures bicarbonate using either an ISO or ISE. In some embodiments, the sensor assembly 146 measure differences in dialysate 28 and serum electrolyte values producing a lagging measure of plasma electrolyte concentrations.

In some embodiments, one or more of: the first sensor 152, the second sensor 154, the third sensor 156, the fourth sensor 158, and the fifth sensor 160 are spectrophotometric or colorimetric sensors that are used by the sensor assembly 146 to measure one or more attributes of the waste fluid 32. In some embodiments, when an ISO sensor is used as one of the sensors, the hue measuring device is also one of the sensors. The hue measuring device may read the hue and displays a number to the patient. In several embodiments, the number is displayed at the fifth sensor 160. In one or more embodiments, the sensor assembly 18 outputs qualitative values rather than quantitative such as by using a color scale.

In one or more embodiments, one or more of: the first sensor 152, the second sensor 154, the third sensor 156, the fourth sensor 158, and the fifth sensor 160 are an anion sensor, which is used to measure phosphate and/or phosphorous. In one or more embodiments, phosphate anions react with chemicals to produce a stable new chemical compound that may absorb light of a specific wavelength, and the absorption peak may correlate to phosphate concentration of the waste fluid 32. In some embodiments, a pre-treatment might be needed to remove colored organic compounds by constructing an appropriate microfluid channel.

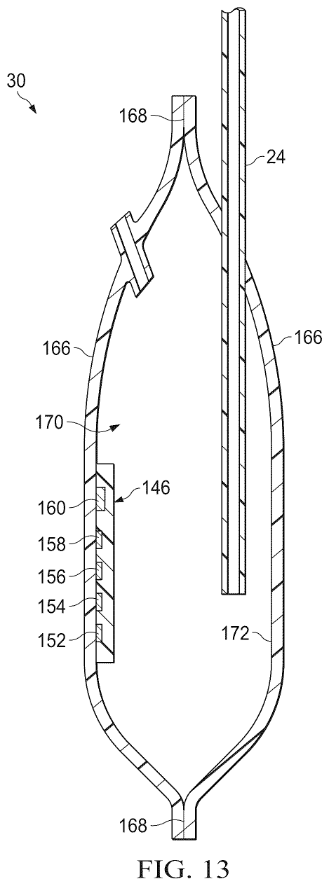

Referring to FIG. 13, one embodiment of the sensor assembly 146 supported on the waste receptacle 30 is shown in more detail and includes at least two exterior walls 166 that are coupled together at welds 168, defining an interior region 170. The sensor assembly 146 is directly coupled to the at least two interior walls 172.

In several embodiments, the waste receptacle 30 include only two exterior walls. In other embodiments, more than two exterior walls are included. In several embodiments, the waste receptacle 30 include only two interior walls. In other embodiments, more than two interior walls are included.

In one or more embodiments, the welds 168 may include stitching. In some embodiments, the at least two exterior walls 166 are coupled together at the welds 168 using heat, pressure, or the like.

In one or more embodiments, the waste fluid 32 is disposed within the interior region 170. In some embodiments, the sensor assembly 146 is disposed within the interior region 170 and coupled to one of the at least two walls 166. In several embodiments, the first sensor 152, the second sensor 154, the third sensor 156, the fourth sensor 158, and the fifth sensor 160 of the sensor assembly 146 extends laterally along a length of the waste receptacle 30.

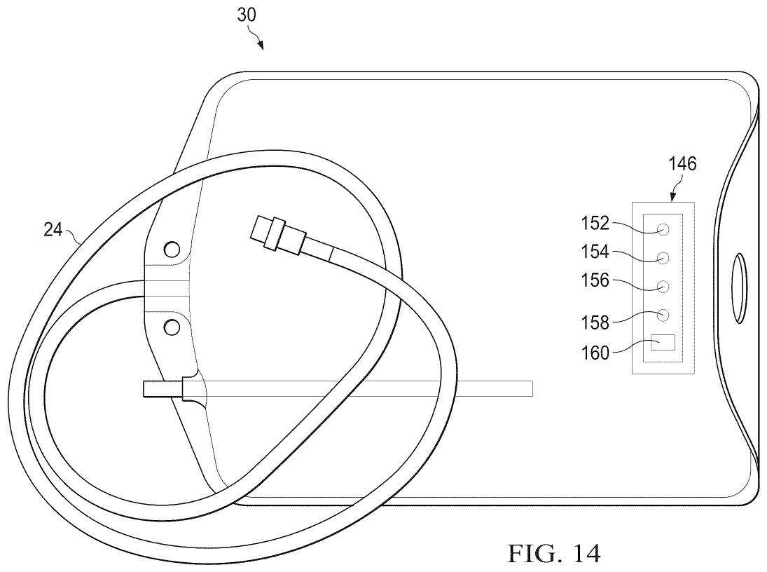

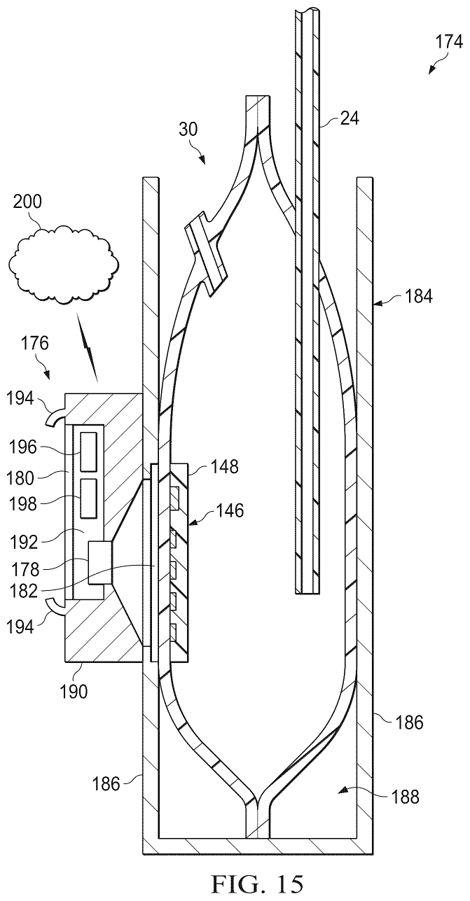

Referring to FIG. 14, one embodiment of the sensor assembly 146 disposed on the waste receptacle 30 is shown in more detail. The first sensor 152, the second sensor 154, the third sensor 156, the fourth sensor 158, and the fifth sensor 160 of the sensor assembly 146 extends horizontally along a width of the waste receptacle 30.

In some embodiments, the first sensor 152, the second sensor 154, the third sensor 156, the fourth sensor 158, and the fifth sensor 160 are disposed near a first end of the waste receptacle 30 opposite a second end to which the waste line 24 is coupled.

Referring to FIG. 15, another embodiment of a PD system 10 is shown and generally referred to by reference numeral 174. In this embodiment, PD system 10 includes a sensor assembly 146 supported on the waste receptacle 30, which is waste receptacle 30 is is shown in more detail and is. The sensor assembly 146 is in communication with smart device 176. The smart device 176 includes a camera 178 and a display 180, disposed on the opposite side of the smart device 176 as the camera 178. The frame 148 of the sensor assembly 146 is coupled to groove 182 of a rigid support structure 184. The rigid support structure 184 extends around at least a portion of the waste receptacle 30. The rigid support structure 184 includes a plurality of walls 186 that define a first interior region 188. The waste receptacle 30 is disposed within the first interior region 188. The rigid support structure 184 also includes a cradle 190 that extends off of one of the plurality of walls 186. The cradle 190 includes a second interior region 192 and a plurality of tabs 194. The smart device 176 is disposed within the second interior region 192 and includes a processor 196 and a memory 198. In some embodiments, the smart device 176 may be linked to a network 200. It will be understood that waste receptacle 30 is typically a non-rigid bag, and thus, in order to position sensor assembly 146 for interaction with smart device 176, waste receptacle 30 is disposed within rigid support structure 184 so that sensor assembly 146 can be positioned adjacent groove 182.

In operation, the waste receptacle 30 is positioned within the first interior region 188 of the rigid support structure 184, and the frame 148 of the sensor assembly 146 is coupled to the groove 182 of the rigid support structure 184. The smart device 176 is positioned within the second interior region 192 and held in place by the plurality of tabs 194. The camera 178 of the smart device 176 is positioned such that the camera may image the sensor assembly 146. Waste fluid 32 fills the waste receptacle 30. The sensor assembly 146 detects a variety of attributes, and the camera 178 images the sensor assembly 146. The processor 196 may process data of the imaged sensor assembly 146 to determine a concentration level of a variety of attributes of the waste fluid 32, and the memory 198 may store the processed data. The display 180 may show the processed data to a patient to inform the patient of the concentration level of the variety of attributes. The smart device 176 may also transmit the processed data via the network 200 to a database or another smart device (not shown).

In one or more embodiments, the sensor assembly 146 includes the camera 178 to image one or more of: the first sensor 152, the second sensor 154, the third sensor 156, the fourth sensor 158, and the fifth sensor 160. In several embodiments, the processor 196 is a microprocessor that determines which analyte/attribute belongs to which sensor by use of a label or order of the sensors. In several embodiments, the processor 196 is a microprocessor that determines which ISO belongs to which analyte/attribute by use of a label or positioning of ISO. In some embodiments, the sensor assembly 146 includes the camera 178 to image an ISO used. In some embodiments, the processor 196 will produce a quantitative value corresponding to the imaged analyte/attribute concentration. In some embodiments, the camera 178 includes a magnifying lens. In some embodiments, the camera 178 is lined up with central carrier 150 (such as a paper carrier). In one or more embodiments, the camera 178 runs for the entire duration of the PD treatment. In several embodiments, the camera 178 takes one or more digital photographs. In other embodiments, the camera 178 records a video. In some embodiments, the camera 178 images the sensor assembly when one or more of: the first sensor 152, the second sensor 154, the third sensor 156, the fourth sensor 158, and the fifth sensor 160 change colors and/or hues.

In several embodiments, the frame 148 includes a groove, slide, or other fastener that mates with groove 182. In several embodiments, the groove 182 is a slide, bump, or other similar fastener.

In one or more embodiments, the smart device 176 is a cellular device and the display 180 forms a portion of a mobile application. In some embodiments, the smart device 176 is a computer, personalized laptop, tablet, desktop computer, set of computers, cellular device (such as a smart phone) or the like. In one or more embodiments, data, processed data, and images created by the smart device 176 are also sent over network 200 to another smart device (such as a patient computer). In some embodiments, the smart device 176 captures digital images or video using the camera 178. In some embodiments, an image of the sensor assembly 146 is captured using the camera 178 of the smart device 176 and sent over the network 200 for processing and analysis at another location and/or by another device. In other embodiments, the patient captures an image of the sensor 42 using the smart device 50 and the smart device 50 processes and analyzes sensing data such as colorimetric data using the processor 196.

In some embodiments, the display 180 shows the image captured by the camera 178. In one or more embodiments, the display 180 depicts a visual indicator to indicate if the smart device 176 is aligned with the sensor assembly 146. In some embodiments, the display 180 shows the captured image, showing the various hues of the one or more sensors (such as the first sensor 152, the second sensor 154, the third sensor 156, the fourth sensor 158, and the fifth sensor 160) and the associated color chart to correlate the color/hue to concentration level of the measured attribute. In some embodiments, the display 180 may display a portion of a patient-facing application that includes electronic health records. In some embodiments, the display 180 displays the processed data. In one or more embodiments, the display 180 displays indicators, which are graphical representations of the attributes. In some embodiments, the display 180 displays a graph showing a change over time of the levels of the attributes. In some embodiments, the display 180 shows changes in peritoneal membrane characteristics. In one or more embodiments, the display 180 shows treatment recommendations such as an optimized treatment plan, which may include changing the composition of dialysate 28. In several embodiments, the display 180 displays a readout of the waste fluid, or spent dialysate, concentrations in mmol, mg/L, or compares the data to normal population serum values or target values input by the patient or the medical professional.

In some embodiments, the rigid support structure 184 surrounds all sides of the waste receptacle 30. In some embodiments, the sensor assembly 146 is positioned at the bottom of the waste receptacle 30, the rigid support structure 184 completely surrounds the waste receptacle 30 except for a hole to image the sensor assembly 146, and the cradle is placed on the bottom of the rigid support structure to image the sensor assembly 146.

In several embodiments, the cradle 190 includes a window, hole or opening that aligns with a window, hole, or opening in the rigid support structure 184 so that the camera 178 may image the sensor assembly 146. In several embodiments, the cradle 190 includes a magnifying lens. In some embodiments, the cradle 190 may include an opening for a charging cord to connect a charging cord to a port on the smart device 176. In some embodiments, the cradle 190 includes a lip to hold the smart device 176, and the plurality of tabs 194 are omitted.

In some embodiments, the processor 196 analyzes hue or color data of each sensor such as first sensor 152, the second sensor 154, the third sensor 156, the fourth sensor 158, and the fifth sensor 160. In one or more embodiments, the processor 196 analyzes and processes image data from the sensor assembly 146 to determine electrolyte levels and/or PH levels and transmits that processed data to the memory 198 and/or display 180 or over the network 200. In several embodiments, the processor 196 determines peritoneal membrane characteristics. In some embodiments, based on the analyzed data, the processor 196 makes treatment recommendations such as an optimized treatment plan, which may include changing the composition of the dialysate 28. In several embodiments, the processor 196 determines electrolyte levels/concentrations or pH levels based on the analysis of processor 196. In some embodiments, the processor 196 stores threshold values or colors of electrolyte concentration and/or PH levels to determine if the concentration and/or levels are normal, low, or high within memory 198. In several embodiments, a plurality of instructions stored on a non-transitory computer readable medium may be executed by processor 196 to cause the processor 196 to carry out or implement in whole or in part the above-described operation of each of the described example embodiments. In one or more example embodiments, the processor 196 may include one or more of the microprocessors, any processor(s) that are part of the components of the system, and/or any combination thereof, and such a computer readable medium may be distributed among one or more components of the system. In several example embodiments, such a processor may execute the plurality of instructions in connection with the smart device 176. In several example embodiments, such a plurality of instructions may communicate directly with the one or more processors, and/or may interact with one or more operating systems, middleware, firmware, other applications, and/or any combination thereof, to cause the one or more processors to execute the instructions.

In one or more embodiments, the memory 198 is in communication with the processor 196. The memory 198 may be accessed by the processor 196 during processing of the imaging data, for example, to pull previously stored image data for comparison with the newly acquired imaging data. In some embodiments, the memory 198 may include random access memory (RAM), flash memory, read only memory (ROM), programmable read only memory (PROM), erasable programmable read only memory (EPROM), and the like.

In some embodiments, the network 200 includes the Internet, one or more local area networks, a Bluetooth® low energy network, one or more wide area networks, one or more cellular networks, one or more wireless networks, one or more voice networks, one or more data networks, one or more communication systems, and/or any combination thereof. In some embodiments, multiple networks are used to transmit data. In some embodiments, the network 200 transmits the image and/or processed data to a database, to a cloud storage, or another smart device such as the medical professional's smart device.

Although several example embodiments have been described in detail above, the embodiments described are examples only and are not limiting, and those skilled in the art will readily appreciate that many other modifications, changes, and/or substitutions are possible in the example embodiments without materially departing from the novel teachings and advantages of the present disclosure. Accordingly, all such modifications, changes, and/or substitutions are intended to be included within the scope of this disclosure as defined in the following claims. In the claims, any means-plus-function clauses are intended to cover the structures described herein as performing the recited function and not only structural equivalents, but also equivalent structures. Moreover, it is the express intention of the applicant not to invoke 35 U.S.C. § 112(f) for any limitations of any of the claims herein, except for those in which the claim expressly uses the word “means” together with an associated function.

1. A peritoneal dialysis sensor assembly comprising:
a disposable sensing array comprising a carrier having a plurality of potentiometric sensors on the carrier and a plurality of microchannels disposed on the carrier, each one of the plurality of sensors disposed adjacent a different each one of the plurality of microchannels, each of the plurality of microchannels having a first end and an opposing second end with a sensor disposed at the second end of each microchannel;
a peritoneal dialysis waste fluid vessel for waste fluid; and
a support housing securing the disposable sensing array adjacent the peritoneal dialysis waste fluid vessel so that the first end of each of the plurality of microchannels is in fluid communication with peritoneal dialysis waste fluid vessel.

2. The peritoneal dialysis sensor assembly of claim 1, wherein cell each of the plurality of sensors and adjacent microchannel is specific to a different one of a plurality of analytes, wherein the plurality of analytes are selected from the group consisting of potassium, glucose, magnesium, sodium, pH, calcium, phosphate, urea, creatine, blood urea nitrogen (BUN), bicarbonate, and lactate.

3. The peritoneal dialysis sensor assembly of claim 2, further comprising a power supply; a microprocessor coupled to the power supply, wherein the power supply and the microprocessor are electronically coupled to each sensor via electrical lines; and a transmitter coupled to the power supply and the microprocessor; wherein the support housing comprises a first housing portion and a second housing portion releasably engageable with one another, wherein the sensing array is disposed within the first housing portion; and the power supply, microprocessor, and transmitter are disposed within the second housing portion.

4. The peritoneal dialysis sensor assembly of claim 1, wherein at least one of the sensors comprises an electrochemical sensor.

5. The peritoneal dialysis sensor assembly of claim 1, wherein at least one of the sensors comprises a sensor configured to detect at least one of: potassium, sodium, calcium, magnesium, phosphate, pH, and glucose.

6. The peritoneal dialysis sensor assembly of claim 1, wherein the peritoneal dialysis waste fluid vessel is a peritoneal dialysis bag and the support housing is a frame carried by the bag.

7. The peritoneal dialysis sensor assembly of claim 1, wherein the carrier comprises paper with the plurality of microchannels formed on the paper; and wherein at least two of the plurality of microchannels have different shapes.

8. The peritoneal dialysis sensor assembly of claim 1, wherein the peritoneal dialysis waste fluid vessel is a rigid conduit to which the support housing is engaged, wherein the rigid conduit defines a waste fluid flow path therethrough, the sensing array having a first end and a second end with a plurality of microchannels extending from adjacent the first end to adjacent the second end, with a plurality of sensors disposed at the first end of the sensing array adjacent the first plurality of microchannels, the first end of the sending array engaged by the support housing, the support housing releasably secured to the rigid conduit so that the second end of the sensing array extends into the waste fluid flow path; the peritoneal dialysis sensor assembly further comprising a power supply; a microprocessor coupled to the power supply, wherein the power supply and the microprocessor are electronically coupled to each sensor; and a transmitter coupled to the power supply and the microprocessor; wherein the power supply, microprocessor, and transmitter are disposed in the support housing.

9. A peritoncal dialysis system, comprising:
a catheter configured for insertion in a peritoneum of a patient;
a peritoneal dialysis waste fluid vessel for waste fluid fluidly coupled to the catheter;
a sensor assembly in fluid communication with the waste fluid vessel;
wherein the sensor assembly comprises a disposable sensing array having a carrier with a plurality of potentiometric sensors disposed on the carrier and a plurality of microchannels disposed on the carrier adjacent the plurality of sensors, wherein each of the plurality of microchannels has a first end and a second end, wherein each of the plurality of microchannels has a different one of the plurality of potentiometric sensors disposed adjacent the second end of each of the plurality of microchannels;
an outer housing supporting the sensing array, wherein the second end of each of the plurality of microchannels is disposed within the outer housing and the first end of each of the plurality of microchannels extends from the outer housing;
and
a housing engaged with the outer housing to secure the disposable sensing array within the outer housing, the housing including a power supply and a microprocessor coupled to the power supply, wherein the power supply and the microprocessor are electronically coupled to each potentiometric sensor via electrical lines when the housing is engaged with the outer housing.

10. The peritoneal dialysis system of claim 9, wherein the peritoneal dialysis waste fluid vessel is a drain line assembly including a drain line assembly vessel having an interior defined thereby; and wherein the first end of each of the plurality of microchannels extends into the interior of the drain line assembly vessel.

11. The peritoneal dialysis system of claim 10, further comprising a transmitter, coupled to the power supply and the microprocessor.

12. A peritoneal dialysis method, comprising:
inserting a catheter into a peritoneum of a patient;
introducing, via the catheter, dialysate from a dialysate bag into the peritoneum;
removing, via the catheter, waste fluid from the peritoneum into a drain line; and
measuring, via a sensor assembly, during removal of the waste fluid, at least two different electrolyte concentrations of a first portion of the waste fluid; wherein measuring comprises introducing a first microchannel into the waste fluid and utilizing a first potentiometric sensor to identify a first electrolyte; and introducing a second microchannel into the waste fluid and utilizing a second potentiometric sensor to identify a first electrolyte.

13. The method of claim 12, further comprising: transmitting, after the measuring, via a network, the at least two electrolyte concentrations to a computer.

14. The method of claim 12, wherein the step of measuring comprises selecting a microchannel disposed to have a capillary force tuned to a desired electrolyte for measurement; positioning the microchannel in a flow stream of waste fluid; and detecting an electrolyte in the microchannel utilizing the potentiometric sensor disposed adjacent the microchannel.

15. The method of claim 12, further comprising:
transmitting, after measuring, via a network, the at least one electrolyte concentration to a computer; and
displaying, via a graphical user interface of the computer, a graphical representation of the at least one electrolyte concentration.

16. The method of claim 12, wherein the at least one electrolyte concentration comprises any one or more of: potassium, magnesium, sodium, calcium, or phosphate.

17. The method of claim 12, further comprising: measuring, via the sensor assembly, a glucose level of the first portion of the waste fluid.

18. The method of claim 12, further comprising: separating the first portion of the waste fluid from a second portion of the waste fluid via a capillary force.

19. The method of claim 12, wherein the sensor assembly comprises:
a plurality of microchannels; and a sensor array, wherein each sensor of the sensor array is disposed at an end of a microchannel of the plurality of microchannels, respectively.

20. The method of claim 19, wherein a first sensor in the sensor array measures an electrolyte concentration of a first electrolyte; and wherein a second sensor in the sensor array measures an electrolyte concentration of a different, second electrolyte.

21. The system of claim 2, wherein the plurality of microchannels comprise a first microchannel tuned to detect a first analyte and a second microchannel tuned to detect a second analyte different than the first analyte, wherein the first microchannel has a first diameter and the second microchannel has a second diameter smaller than the first diameter.

22. The system of claim 2, wherein the plurality of microchannels comprise a first microchannel tuned to detect a first analyte and a second microchannel tuned to detect a second analyte different than the first analyte, wherein the first microchannel has a first length and the second microchannel has a second length shorter than the first length.

23. The system of claim 2, wherein the plurality of microchannels comprise a first microchannel tuned to detect a first analyte and a second microchannel tuned to detect a second analyte different than the first analyte, wherein the first microchannel has a first geometry and the second microchannel has a second geometry different than the first geometry.
